(19) United States
(12) Patent Application Publication
(10) Pub. No.: US 2015/0273088 A1
(43) Pub. Date: Oct. 1, 2015

(54) ZAPRINAST ANALOGUES AS GLUTAMINASE INHIBITORS AND METHODS TO PREDICT RESPONSE THERETO

(71) Applicants: Board of Regents, The University of Texas System, Austin, TX (US); Washington University, St. Louis, MO (US)

(72) Inventors: David PIWNICA-WORMS, Houston, TX (US); Adnan ELHAMMALI, St. Louis, MO (US); Joseph E. IPPOLITO, St. Louis, MO (US); Seth GAMMON, Houston, TX (US)

(73) Assignees: WASHINGTON UNIVERSITY, St. Louis, MO (US); BOARD OF REGENTS, THE UNIVERSITY OF TEXAS SYSTEM, Austin, TX (US)

(21) Appl. No.: 14/669,700

(22) Filed: Mar. 26, 2015

Related U.S. Application Data
(60) Provisional application No. 61/971,877, filed on Mar. 28, 2014.

(51) Int. Cl. A61K 51/04; A61K 49/10; C07D 487/04

(52) U.S. Cl. CPC A61K 51/0459; C07D 487/04; A61K 49/10

(57) ABSTRACT

Zaprinast has been discovered to have activity against glutaminase 1, an important metabolic enzyme in selected cancers, for example, glutamine-dependent cancer types. Thus, glutamine-dependent cancer types, such as IDH1/2 gain-of-function mutant cancers or GLI1 overexpressing cancers, may be particularly sensitive to Zaprinast analogues.

The present application claims the priority benefit of U.S. provisional application No. 61/971,877, filed Mar. 28, 2014, the entire contents of which is incorporated herein by reference.

The invention was made with government support under Grant No. P50 CA094056 awarded by the National Institutes of Health. The government has certain rights in the invention.

### BACKGROUND OF THE INVENTION

1. Field of the Invention

The present invention relates generally to the fields of biochemistry, cancer biology, and neurobiology. More particularly, it concerns methods of inhibiting glutaminase (GLS1) to decrease 2-hydroxyglutarate in the context of cancer and to affect glutamine:glutamate ratios in the context of neurodegenerative diseases.

2. Description of Related Art

Altered glutamine metabolism can maintain oncogenic transformation and support rapid growth in some cancer cells (DeBerardinix and Cheng, 2010). Oncogenic MYC regulates glutamine metabolism by increasing both the uptake of glutamine and its catabolism through microRNA-driven regulation of glutaminase (GLS1). As such, MYC-transformed cells are dependent on glutamine for growth (Wise et al., 2008; Gao et al., 2009). Additionally, RAS-driven reprogramming of cellular metabolism shunts glutamine toward NADPH-generating reactions to maintain oxidative balance (Son et al., 2013). Glutamine, through glutamate, is also a precursor for cellular α-ketoglutarate (αKG), which can undergo further metabolism through the Krebs cycle or can be further metabolized to 2-hydroxyglutarate (2HG) by mutant isocitrate dehydrogenase (IDH) (Dang et al., 2009). Additionally, over-expression of glutaminase alone has been shown to be sufficient to increase 2HG in breast cancer even in the context of wild-type IDH (Terunuma et al., 2014). Thus, inhibition of glutaminase may be important in a wide variety of cancers including those with wild-type IDH but that exhibit increased 2HG. Finally, as of yet underscovered mutations in glutaminase may also increase the availability of 2HG and be a therapeutic target for glutaminase inhibitors.

Altered glutamine regulation through either upregulation of glutaminase or through glutaminase mutants also affects the glutamine:glutamate ratio, which is important for neuronal communication in the brain. Indeed, HIV has been shown to upregulate glutaminase, and the inhibition with siRNA and different classes of small molecules reduced neurotoxicity. In addition, heterozygous knock out mice for glutaminase show significant resistance to schizophrenia. More broadly any psychiatric disorder where the glutamine:glutamate ratio is too low may be a glutaminase inhibitor target (Ongur et al., 2011).

Heterozygous somatic mutations in IDH enzymes are present in over 80% of grade II and III gliomas as well as secondary glioblastomas (Parsons et al., 2008; Yan et al., 2009). Mutations have also been detected in acute myeloid leukemia, chondrosarcomas, and cholangiocarcinoma among others (Mardis et al., 2009; Amary et al., 2001; Borger et al., 2012). Nearly all identified mutations are in arginine residues 100 and 132 of IDH1 or residues 140 and 172 of IDH2, all of which are located in the active sites of the enzymes (Losman and Kaelin, 2013). Instead of inhibiting the enzymatic activity of IDH, these mutations alter the catalytic activity such that the normal product, αKG, is metabolized to R-2HG in a reaction that consumes NADPH. While endogenous levels of 2HG are normally low, gliomas harboring mutant IDH1 or IDH2 accumulate millimolar quantities of R-2HG (Dang et al., 2009).

Structural similarities between 2HG and αKG suggested that 2HG could modulate the function of αKG-dependent dioxygenases to promote transformation and alter differentiation. Indeed, 2HG inhibits the TET family of methylcytosine dioxygenases as well as several members of the JmjC family of histone demethylases (Xu et al., 2011; Figueroa et al., 2010). In addition, R-2HG stimulates EglN, driving the degradation of hypoxia inducible factor (HIF) (Koivunen et al., 2012). Modulating the activity of these various dioxygenases drives DNA and histone hypermethylation, blocks differentiation, and promotes transformation (Lu et al., 2012; Losman et al., 2013; Sasaki et al., 2012). Importantly, some of these events are reversible. Withdrawal of cell permeable 2HG or treatment with a small molecule inhibitor targeting IDH2 R140Q restores differentiation of leukemia cells, while inhibition of IDH1 R132H in transformed cells reduces histone methylation and soft-agar growth (Losman et al., 2013; Rohle et al., 2013; Wang et al., 2013). Thus, reducing 2HG could provide therapeutic benefit in patients with malignancies harboring gain-of-function IDH mutations.

### SUMMARY OF THE INVENTION

Gain-of-function IDH mutations are common events in glioma, AML, and other cancer types, which lead to the accumulation of the onco-metabolite 2HG. The drug Zaprinast is capable of reducing cellular 2HG levels by inhibiting the upstream enzyme GLS1, thus identifying a new strategy to target 2HG production in selected cancers and psychiatric disorders.

In one embodiment, provided herein is a compound of the formula:

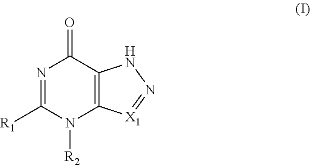

wherein:
R₁ and R₂ are each independently selected from hydrogen, alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, haloalkoxy_{(C≦12)}, alkenyloxy_{(C≦12)}, alkynyloxy_{(C≦12)}, aryloxy_{(C≦12)}, aralkyloxy_{(C≦12)}, heteroaryloxy_{(C≦12)}, heteroaralkyloxy_{(C≦12)}, acyloxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or

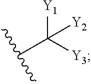

wherein:
   Y₁ and Y₂ are independently selected from alkoxy_{(C≦8)} or substituted alkoxy_{(C≦8)} or Y₁ and Y₂ are taken together and are alkoxydiyl_{(C≦8)} or substituted alkoxydiyl_{(C≦8)}; and
      Y₃ is hydrogen, alkyl_{(C≦8)}, or substituted alkyl_{(C≦8)}; and
X₁ is N or CR₃;
   R₃ is selected from hydrogen, alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, haloalkoxy_{(C≦12)}, alkenyloxy_{(C≦12)}, alkynyloxy_{(C≦12)}, aryloxy_{(C≦12)}, aralkyloxy_{(C≦12)}, heteroaryloxy_{(C≦12)}, heteroaralkyloxy_{(C≦12)}, acyloxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or

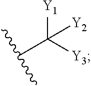

wherein:
   Y₁ and Y₂ are independently selected from alkoxy_{(C≦8)} or substituted alkoxy_{(C≦8)}, or Y₁ and Y₂ are taken together and are alkoxydiyl_{(C≦8)} or substituted alkoxydiyl_{(C≦8)}; and
   Y₃ is hydrogen, alkyl_{(C≦8)}, or substituted alkyl_{(C≦8)};
      or a pharmaceutically acceptable salt, tautomer, acetal, or ketal thereof. In some aspects, for formula I is not Zaprinast.

In one aspect, the compound may be further defined as:

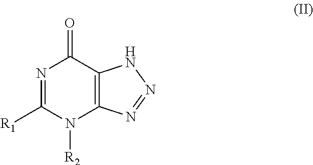

wherein:
R₁ and R₂ are each independently selected from hydrogen, alkyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, haloalkoxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or

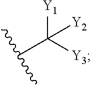

wherein:
   Y₁ and Y₂ are independently selected from alkoxy_{(C≦8)} or substituted alkoxy_{(C≦8)} or Y₁ and Y₂ are taken together and are alkoxydiyl_{(C≦8)} or substituted alkoxydiyl_{(C≦8)}; and
   Y₃ is hydrogen, alkyl_{(C≦8)}, or substituted alkyl_{(C≦8)};
      or a pharmaceutically acceptable salt, tautomer, acetal, or ketal thereof.

In a further aspect, the compound may be further defined as:

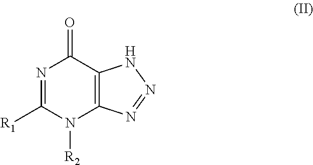

wherein:
R₁ and R₂ are each independently selected from hydrogen, alkyl_{(C≦12)}, alkoxy_{(C≦12)}, haloalkoxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or

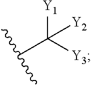

wherein:
   Y₁ and Y₂ are independently selected from alkoxy_{(C≦8)} or substituted alkoxy_{(C≦8)}; and
   Y₃ is hydrogen, alkyl_{(C≦8)}, or substituted alkyl_{(C≦8)};
      or a pharmaceutically acceptable salt, tautomer, acetal, or ketal thereof.

In some aspects, R₁ may be arenediyl_{(C≦12)}-alkoxy_{(C≦8)} (e.g., 2-propoxyphenyl). In some aspects, R₂ may be hydrogen.

In certain aspects, the compound may be further defined as:

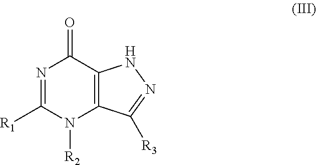

wherein:
R₁, R₂, and R₃ are each independently selected from hydrogen, alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, haloalkoxy_{(C≦12)}, alkenyloxy_{(C≦12)}, alkynyloxy_{(C≦12)}, aryloxy_{(C≦12)}, aralkyloxy_{(C≦12)}, heteroaryloxy_{(C≦12)}, heteroaralkyloxy_{(C≦12)}, acyloxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or

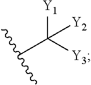

wherein:
   Y₁ and Y₂ are independently selected from alkoxy_{(C≦8)} or substituted alkoxy_{(C≦8)} or Y₁ and Y₂ are taken together and are alkoxydiyl_{(C≦8)} or substituted alkoxydiyl_{(C≦8)}; and
   Y₃ is hydrogen, alkyl_{(C≦8)}, or substituted alkyl_{(C≦8)};
      or a pharmaceutically acceptable salt, tautomer, acetal, or ketal thereof.

In some aspects, the compound may be further defined as:

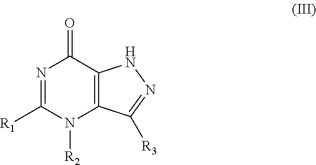

wherein:
R₁, R₂, and R₃ are each independently selected from hydrogen, alkyl_{(C≦12)}, alkoxy_{(C≦12)}, haloalkoxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or

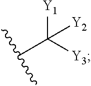

wherein:
   Y₁ and Y₂ are independently selected from alkoxy_{(C≦8)} or substituted alkoxy_{(C≦8)}; and
      Y₃ is hydrogen, alkyl_{(C≦8)}, or substituted alkyl_{(C≦8)};
         or a pharmaceutically acceptable salt, tautomer, acetal, or ketal thereof.

In various aspects, R₁ may be -arenediyl_{(C≦12)}-alkoxy_{(C≦8)} (e.g., propoxyphenyl).

In further aspects, the compounds according to the embodiments are provided in one or more sealed container(s) and/or are formulated in pharmaceutically acceptable carrier. In certain aspects, compounds of the embodiments are between about 90% and about 99.9% pure. In certain embodiments, the compounds set forth herein are about or at least about 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9% pure.

In a further embodiment there is provided a compound of the embodiments (e.g., a compound having formula I, II or III), wherein the compound comprises a heavy-isotope or radionuclide label. For example, in some aspects, a compound of the embodiments comprises a ¹¹C, ¹³N or ¹⁵O substituted at one or more of the positions of the compound. For example, in further aspects, a compound of the embodiments comprises a ¹³C, ¹⁵N, or ¹⁸O substituted at one or more of the positions of the compound for readout by mass spectrometry or hyperpolarization. In still further aspects a compound of the embodiments comprises a group including radionuclide label. For example, the radionuclide can be a radiohalide, such as ¹⁸F, ⁷⁵Br, ⁷⁶Br, ¹²⁴I, ¹²⁵I or ¹³¹I. Further examples of radionuclides include, but are not limited to, ^{99m}Tc, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²¹²Bi, ²¹¹At, ⁴⁵Ti, ⁶⁰Cu, ⁶¹Cu, ⁶⁷Cu, and ⁶⁴Cu, In still further aspects, an imagining composition is provided comprising a heavy-isotope-labeled compound of the embodiments formulated in a pharmaceutically acceptable carrier.

In some embodiments, a method is provided for treating cancer in a patient in need thereof comprising administering to the patient a therapeutically effective amount of a compound of the present embodiments (e.g., a compound having formula I, II or III) or a compound of the formula:

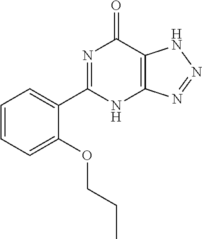 [SMILES: CCCOc1ccccc1-c1nc(=O)c2[nH]nnc2[nH]1]

or a pharmaceutically acceptable salt or tautomer thereof. In still further aspects, a method of the embodiments can comprise administering to a patient a compound as described in U.S. Pat. Nos. 6,156,753 or 5,521,191 (incorporated herein by reference).

In some aspects, the cancer may have been determined to have an IDH1 or IDH2 mutation. In some aspects, the method may further comprise selecting a patient determined to comprise a cancer comprising an IDH1 or IDH2 mutation prior to the administering step. In some aspects, the method may further comprise obtaining a sample of the cancer and determining if the cancer comprises an IDH1 or IDH2 mutation prior to the administering step. In some aspects, the patient may have been identified as having a cancer with elevated levels of 2-hydroxyglutarate (2HG). In some aspects, the patient may have been identified as having a cancer that overexpresses glutaminase. In some aspects, the patient may have been identified as having a cancer that comprises hyperactivated glutaminase (i.e., a mutated glutaminase having elevated enzymatic activity).

In some aspects, the cancer may have increased flux through the glutaminase pathway. This may be determined using noninvasive imaging of hyperpolarization of heavy isotope, ¹⁵N or ¹³C, labeled glutamine, glutamate, alpha-ketoglutarate, or Zaprinast analogues. In other aspects this may be determined using invasive mass spectrometry analysis or imaging of biopsies, tissue, or blood samples.

In various aspects, the IDH1 mutation may be a mutation at amino acid 100 or 132 of the IDH1 protein (NCBI Reference Sequence NP_{—}005887). In certain aspects, the mutation at amino acid 132 of the IDH1 protein may be selected from the group consisting of R132H, R132C, R132S, R132G, and R132L.

In various aspects, the IDH2 mutation may be a mutation at amino acid 140 or 172 of the IDH2 protein (NCBI Reference Sequence NP_{—}002159). In certain aspects, the mutation at amino acid 140 or 172 of the IDH2 protein may be selected from the group consisting of R140Q, R172M, R172K, and R172G.

In some aspects, the amino acid present at position 100 or 132 of the IDH1 protein or at position 140 or 172 of the IDH2 protein may be determined by mass spectrometry, western blot, ELISA, or sequencing a nucleic acid comprising at least a portion of the protein coding sequence of the IDH1 or IDH2 protein.

In some aspects, the method may further comprise providing a report (e.g. a written or electronic report) of the determining. In some aspects, the method may further comprise providing the report to the patient, a health care payer, a physician, an insurance agent, or an electronic system.

In some aspects, the cancer may be metastatic, recurrent, or multi-drug resistant. In various aspects, the cancer may be a glioma, glioblastoma, acute myeloid leukemia, cholangiocarcinoma, chondrosarcoma, colorectal cancer, or pancreatic cancer. In some aspects, the cancer may be a carcinoma, sarcoma, lymphoma, leukemia, melanoma, mesothelioma, multiple myeloma, or seminoma. In other embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some aspects, the patient may be a human. In other aspects, the patient may be a non-human mammal.

In certain aspects, the therapeutically effective amount may be administered in a single dose per day. In other aspects, the therapeutically effective amount may be administered in two or more doses per day. In some aspects, the compound may be administered systemically. In some aspects, the compound may be administered orally, intraarterially, intramuscularly, intravenously, or intratumorally.

In certain aspects, the patient may be treated at least a second time. In some aspects, the patient may be treated over a period of 1 week to 6 months.

In some aspects, the method may further comprise administering a second therapy. The second therapy may comprise administering a therapeutically effective amount of a second drug to the patient. In some aspects, the second therapy may comprise administering a second anticancer therapy to the patient. The second anticancer therapy may be a surgical therapy, chemotherapy, radiation therapy, cryotherapy, hormonal therapy, toxin therapy, immunotherapy, or cytokine therapy.

Additional combination treatment therapy is also contemplated for the various embodiment of the present disclosure. For example, in some aspects, the methods of treating cancer in a subject, comprising administering to the subject a pharmaceutically effective amount of a compound of the present disclosure, the methods may further comprise one or more treatments selected from the group consisting of administering a pharmaceutically effective amount of a second drug, radiotherapy, immunotherapy, gene therapy, and surgery. In some embodiments, the methods may further comprise (1) contacting a tumor cell with the compound prior to contacting the tumor cell with the second drug, (2) contacting a tumor cell with the second drug prior to contacting the tumor cell with the compound, or (3) contacting a tumor cell with the compound and the second drug at the same time. The second drug may, in certain embodiments, be an antibiotic, anti-inflammatory, anti-neoplastic, anti-proliferative, anti-viral, immunomodulatory, or immunosuppressive. In other embodiments, the second drug may be an alkylating agent, androgen receptor modulator, cytoskeletal disruptor, estrogen receptor modulator, histone-deacetylase inhibitor, HMG-CoA reductase inhibitor, prenyl-protein transferase inhibitor, retinoid receptor modulator, topoisomerase inhibitor, or tyrosine kinase inhibitor. In certain embodiments, the second drug is 5-azacitidine, 5-fluorouracil, 9-cis-retinoic acid, actinomycin D, alitretinoin, all-trans-retinoic acid, annamycin, axitinib, belinostat, bevacizumab, bexarotene, bosutinib, busulfan, capecitabine, carboplatin, carmustine, CD437, cediranib, cetuximab, chlorambucil, cisplatin, cyclophosphamide, cytarabine, dacarbazine, dasatinib, daunorubicin, decitabine, docetaxel, dolastatin-10, doxifluridine, doxorubicin, doxorubicin, epirubicin, erlotinib, etoposide, gefitinib, gemcitabine, gemtuzumab ozogamicin, hexamethylmelamine, idarubicin, ifosfamide, imatinib, irinotecan, isotretinoin, ixabepilone, lapatinib, LBH589, lomustine, mechlorethamine, melphalan, mercaptopurine, methotrexate, mitomycin, mitoxantrone, MS-275, neratinib, nilotinib, nitrosourea, oxaliplatin, paclitaxel, plicamycin, procarbazine, semaxanib, semustine, sodium butyrate, sodium phenylacetate, streptozotocin, suberoylanilide hydroxamic acid, sunitinib, tamoxifen, teniposide, thiopeta, tioguanine, topotecan, TRAIL, trastuzumab, tretinoin, trichostatin A, valproic acid, valrubicin, vandetanib, vinblastine, vincristine, vindesine, or vinorelbine.

In a further embodiment, a method is provided for inhibiting glutaminase in a cell comprising treating the cell with a compound of the embodiments (e.g., a compound having formula I, II, or III) or a compound of the formula:

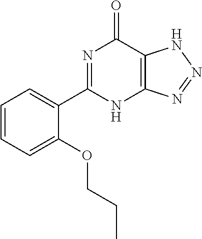 [SMILES: CCCOc1ccccc1-c1nc(=O)c2[nH]nnc2[nH]1]

or a pharmaceutically acceptable salt or tautomer thereof. In further aspects, a method of the embodiments comprises inhibiting glutaminase in patient comprising administering an effective amount of a compound of the embodiments (e.g., a compound having formula I, II, or III) to a patient in need thereof (such as a patient determined to have a disease resulting from elevated glutaminase expression or activity).

In one embodiment, a method is provided for selecting a drug therapy for a cancer patient comprising (a) obtaining a sample of the cancer; (b) determining the presence of a mutation in the IDH1 or IDH2 protein expressed in the cancer; and if a mutation is determined to be present in the IDH1 or IDH2 protein expressed in the cancer, then (c) selecting a compound of the present embodiments (e.g., a compound having formula I, II, or III) or a compound of the formula:

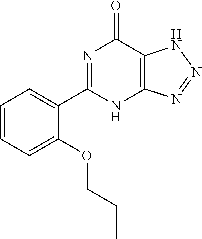 [SMILES: CCCOc1ccccc1-c1nc(=O)c2[nH]nnc2[nH]1]

or a pharmaceutically acceptable salt or tautomer thereof.

In a further aspect, the method may further comprise administering a therapeutically effective amount of a compound of step (c) to the patient. In some aspects, the mutation in the IDH1 protein may be a R132H or R132C mutation. In some aspects, the mutation in the IDH2 protein may be a R140Q or R172M mutation.

In a further embodiment there is provided a method of imaging a patient comprising administering to the patient an effective amount of a compound of the embodiments (e.g., a compound having formula I, II, or III), wherein the compound comprises a heavy-isotope-label. For example, in some aspects, the compound comprises a ¹¹C, ¹³N, or ¹⁵O substituted at one or more of the positions of the compound. In still further aspects, the compound comprises a group including a radionuclide. Thus, in some aspects, a method of imaging a patient comprises (i) administering a compound of the embodiments comprising a heavy-isotope-label to the patient; and (ii) detecting the compound in the patient to produce an image. In certain aspects, a method for imaging a patient may be further defined as a method for diagnosing a subject or a method for detecting a disease in a subject (e.g., by detecting elevated glutaminase expression). Imaging methods for use according to the embodiments include, but are not limited to, hyperpolarized MRI, single photon emission computed tomography (SPECT), positron emission tomography (PET), SPECT/CT, SPECT/MRI, PET/CT and PET/MRI.

In yet a further embodiment, a method is provided for selecting a drug therapy for a cancer patient comprising determining the flux through the glutamine:glutamate pathway and selecting a compound of the present embodiments or Zaprinast if the flux is determined to be higher than a reference level (e.g., the level in a healthy control subject). In some aspects, the flux can be determined by performing hyperpolarized MRI with a heavy-isotope-labeled glutamine or glutamate as the imaging agent. In some aspects, the flux can be determined by obtaining a sample from the patient and performing mass spectrometry with heavy-isotope-labeled glutamate or glutamine.

In still a further embodiment, a method is provided for determining the flux within the glutamate:glutamine pathway comprising performing hyperpolarized MR imaging with a heavy-isotope-labeled glutamine or glutamate as the imaging agent.

In yet a further embodiment, a method is provided for treating a patient with a psychiatric disorder comprising administering to the patient a therapeutically effective amount of a compound of the present embodiments or Zaprinast. In some aspects, the psychiatric disorder can be any psychiatric disorder with 2HG accumulation or increased glutamine:glutamate ratios, such as, for example, bipolar manic disorder, schizophrenia, or HIV-related dementia.

Another general embodiment of the present disclosure is a method of inhibiting GLS1 comprising administering a therapeutically effective amount of a compound of the present embodiments or Zaprinast to a subject in need thereof.

Yet another general aspect of the present disclosure contemplates a method of reducing 2HG levels in a subject in need thereof comprising administering a therapeutically effective amount of a compound of the present embodiments or Zaprinast to the subject.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-G. Microplate assay detects changes in 2HG levels. FIG. 1A. Assay schematic showing the coupling of NADH production by PHGDH to resorufin production by mitochondrial diaphorase. Resorufin accumulates in proportion to 2HG metabolite turnover. FIG. 1B. Accumulation of resorufin with increasing 2HG dissolved in buffer produces a pink solution. FIG. 1C. Quantification of assay fluorescence of 2HG dissolved in complete culture media showing linearity up to 100 μM. Fluorescence is plotted as Arbitrary Units (A.U.). FIGS. 1D-F. Western blot showing overexpression of exogenous IDH1 in NHA cells (FIG. 1D) and HEK293T cells (FIG. 1F) and quantification of 2HG by (FIG. 1D) GC-MS and (FIGS. 1E,F) the fluorimetric assay. FIG. 1G. Western blot of shRNA-mediated knockdown of IDH1 in HT1080 cells with endogenous mutant IDH1 as well as corresponding fluorescent assay signal. 2HG GC-MS values were obtained from cell extracts and were normalized to internal standard and total protein, while the 2HG fluorescent assay was performed on conditioned cell culture media and normalized to cell viability as quantified by Alamar Blue. (n=3, **P<0.05, two-tailed Student's t-test).

FIGS. 2A-E. Zaprinast lowers 2HG levels in IDH1 mutant cells in culture and tumors in vivo. FIG. 2A. HT1080 high-throughput screen results plotted as 2HG vs viability. Data points represent the average of screens performed in triplicate. Linear fit and 95% predictive interval (P.I.) show a correlation between 2HG and viability. Compounds in the top left quadrant outside the 95% P.I. produced a reduction in 2HG that was not predicted to be a consequence of general drug toxicity. FIG. 2B. HT1080 cells were treated with the indicated concentrations of Zaprinast for 48 hours and secreted 2HG was measured in the media using the fluorescent assay (n=3; data are plotted as fold-viability over fold-untreated). Top line is DMSO; bottom line is Zaprinast. FIGS. 2C,D. HT1080 cells (FIG. 2C) and NHA cells (FIG. 2D) ectopically expressing IDH1 R132H treated with Zaprinast for 48 hours were analyzed for intracellular 2HG by GC-MS (n=3). FIG. 2E. GC-MS quantification of 2HG in HT1080 tumor xenografts treated with vehicle or Zaprinast (n=6 for vehicle and n=7 for drug; error bars represent S.E.M; *P<0.05).

FIGS. 3A-E. Effects of Zaprinast on 2HG are not cGMP mediated. HT1080 cells treated with (FIG. 3A) PDE5 inhibitors Sildenafil or Tadalafil, or (FIG. 3B) 8-bromo-cAMP or 8-bromo-cGMP, were analyzed for secreted 2HG (n=3; data are plotted as fold-viability over fold-untreated). FIG. 3C. Western blot of 6× His tag in HT1080 cells expressing sGCα1/βcys105. FIG. 3D. cGMP levels as measured by ELISA in HT1080 cells expressing vector or sGCα1/βcys105 plasmids (n=3; **P<0.05). FIG. 3E. GC-MS of 2HG in extracts of HT1080 cells expressing vector or sGCα1/βcys105 plamids (n=6, error bars represent S.E.M.). C.A., constitutively active.

FIGS. 4A-G. Zaprinast inhibits GLS1. FIG. 4A. Metabolic pathway of glutamine metabolism to 2HG. Abbreviations are as follows: Gln, glutamine; GLS1, glutaminase; Glu, gluamate; GLUD1, glutamate dehydrogenase; αKG, α-ketoglutarate; IDH1, isocitrate dehydrogenase; 2HG, 2-hydroxyglutarate. FIG. 4B. GC-MS quantification of 2HG and upstream metabolites in HT1080 and NHA cells expressing IDH1 R132H treated for 48 hours with Zaprinast. FIG. 4C. GC-MS quantification of 2HG and upstream metabolites in HT1080 cells treated for 48 hours with 300 μM Zaprinast or 10 μM BPTES. FIG. 4D. GC-MS quantification of 2HG and αKG in HT1080 cells treated with Zaprinast alone or with 5 mM cell permeable dimethyl 2-oxoglutarate. Values in panels b-d are normalized to vehicle treatment. FIG. 4E. Enzyme activity of purified, full length, human GLS1 exposed to Zaprinast. FIG. 4F. Kinetic activity of human GLS1 exposed to varying concentrations of Zaprinast and glutamine. FIG. 4G. Double-reciprocal Lineweaver-Burk plot of data from panel f showing noncompetitive inhibition of GLS1 by Zaprinast with respect to glutamine. n=3 or 4, error bars represent S.D.; *P<0.05, **P<0.01, two-tailed Student's t-test.

FIGS. 5A-D. Zaprinast reverses 2HG-associated phenotypes. FIG. 5A. Immortalized NHA cells were transduced with vector, wild type IDH1, or IDH1 R132H plasmids and treated with vehicle or Zaprinast at the indicated concentrations. Histone extraction was then performed. A representative Western blot is shown to assess the levels of indicated histone lysine residues. FIG. 5B. Quantification of histone lysine methylation levels from Western blots performed in three independent experiments. Data were normalized to total H3 and vector control. FIG. 5C. Soft agar colony assay of NHA cells expressing vector, wild type IDH1, or IDH1 R132H plasmids and treated with vehicle or Zaprinast. FIG. 5D. Quantification of triplicate colony assay experiments. n=3, error bars represent S.D.; **P<0.05, two-tailed Student's t-test.

FIGS. 6A-G. Zaprinast inhibits growth of PDAC cells and sensitizes them to oxidative stress. FIG. 6A. Growth of PDAC cells Panc1 and MiaPaca2 in glutamine-free media or in the presence of increasing concentrations of glutamine. FIG. 6B. Relative levels of glutamine, glutamate, and αKG in PDAC cells treated with 300 μM Zaprinast or vehicle control for 48 hours (normalized to cellular protein). Left bar of each pair is DMSO; right bar of each pair is Zaprinast. FIG. 6C. Growth of PDAC cells treated with Zaprinast at the indicated concentrations. Top lines are 0 μM Zaprinast; middle lines are 100; and bottom lines are 300. FIG. 6D. Growth of PDAC cells grown in complete culture media containing 2 mM glutamine. Cells were treated with vehicle or Zaprinast alone or in the presence of 5 mM glutamate. FIG. 6E. Microscopic images of DCFDA-positive PDAC cells following treatment with the indicated concentrations of Zaprinast. FIG. 6F. Quantification of DCFDA-positive PDAC cells. FIG. 6G. PDAC cells treated with vehicle or Zaprinast in the absence or presence of 5 mM glutamate were exposed to increasing concentrations of H₂O₂. Cell viability was assessed with Alamar Blue and values were normalized to vehicle-treated cells. Bottom lines are Zap. n=3, error bars represent S.D.; *P<0.05, **P<0.01; two-tailed Student's t-test used in FIGS. 6B,D,F, while two-way ANOVA was applied to FIGS. 6C,G.

FIG. 7. Full 2HG titration curve using a coupled fluorimetric assay. A 20 μL aliquot of 2HG pulsed into media was quantified by fluorescence following acid-base neutralization. Assay signal saturates at 2HG concentrations of 1 mM. Fluorescence is plotted as Arbitrary Units (A.U.).

FIG. 8. The coupled fluorimetric assay activity is unaffected by metabolites upstream of 2HG. Physiologic concentrations of metabolites upstream of 2HG and part of the glutamine metabolic pathway were assayed alone or in the presence of 2HG at physiologic concentrations (2HG 1 mM, Glutamine 4 mM, Glutamate 4 mM, Isocitrate 50 μM, and α-Ketoglutarate 100 μM).

FIG. 9. cGMP does not inhibit GLS1 activity. Enzyme activity of purified, full length, human glutaminase treated with increasing amounts of cGMP in the presence of 10 mM glutamine.

FIGS. 10A-B. Immortalized NHA cells were transduced with vector, wild type IDH1, or IDH1 R132H plasmids and treated with vehicle or 300 μM Zaprinast. Histone extraction was then performed. Western blot was performed to assess the levels of histone lysine residues H3K9Me2 (FIG. 10A) and H3K9Me3 (FIG. 10B) (top) with band quantification (bottom).

FIG. 11. Effects of glutaminase (GLS1) inhibition on 3-phosphoglycerate levels in HT1080 cells. Quantification of 3-phosphoglycerate levels using GC-MS in HT1080 cells treated with DMSO, 300 μM Zaprinast, or 10 μM BPTES. Values were normalized first to total protein and then to DMSO treatment group to obtain relative metabolite levels (n=4, error bars represent S.D.).

### DESCRIPTION OF ILLUSTRATIVE EMBODIMENTS

Recently identified IDH mutations lead to the production of 2-hydroxyglutarate (2HG), an onco-metabolite aberrantly elevated in selected cancers. A facile and inexpensive fluorimetric microplate assay was developed, as described herein, for quantitation of 2HG and used to perform an unbiased small molecule screen in live cells to identify compounds capable of perturbing 2HG production. It was reasoned that this approach could identify novel drug interactions that ultimately lead to diminished mutant IDH1 activity, and perhaps perturb other related vital pathways such as glutamine metabolism, with the potential to repurpose drugs originally developed for alternative disorders. After validation and optimization of assay conditions, an unbiased screen of a small molecule library was performed using HT1080 cells, which harbor an endogenous IDH1 R132C mutation. Zaprinast, a phosphodiesterase 5 (PDE5) inhibitor, was identified as an efficacious modulator of 2HG production and was confirmed to lower 2HG levels in vivo. The mechanism of action was not due to cGMP stabilization, but rather, profiling of metabolites upstream of mutant IDH1 pointed to targeted inhibition of the enzyme glutaminase (GLS1). Zaprinast treatment reversed histone hypermethylation and soft agar growth of IDH1 mutant cells, and treatment of glutamine-addicted pancreatic cancer cells reduced growth and sensitized cells to oxidative damage. Thus, Zaprinast is efficacious against glutamine metabolism and further establishes the therapeutic linkages between GLS1 and 2HG-mediated oncogenesis.

### I. ASPECTS OF THE PRESENT INVENTION

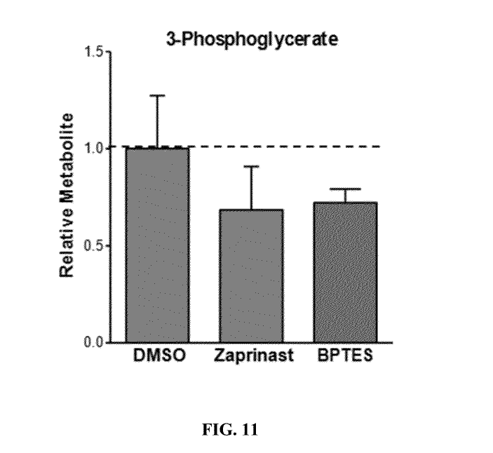

Accumulation of 2HG in tumors harboring IDH1 and IDH2 mutations modulates the activity of several αKG-dependent dioxygenases and leads to histone and DNA hypermethylation, blocked differentiation, and cellular transformation (Losman and Kaelin, 2013). Herein, a fluorimetric assay capable of detecting changes in 2HG levels was developed and found to allow for rapid, high-throughput quantification when compared to mass spectrometry. The present objective was to identify alternative cellular targets or mechanisms for reducing total cellular 2HG levels. An assay using (D)-2-hydroxyglutarate dehydrogenase as the driver enzyme was recently published by Balss et al. to quantify 2HG in patient serum and tumor tissues with mutant IDH (Balss et al., 2012). Compared to E. coli PHGDH, whose primary substrate is 3-phosphoglycerate (3PG), (D)-2-hydroxyglutarate dehydrogenase is thermodynamically more specific for 2HG and could provide an alternative driver enzyme for future experiments. Nonetheless, glutaminase inhibition with either BPTES or Zaprinast did not cause an elevation in cellular 3PG, thus reducing the likelihood of interference within the context of the PHGDH-based assay signals (FIG. 11). The development and optimization of the fluorimetric assay was focused on high-throughput robotic screening and led to the successful screening of a library of 480 bioactive compounds against live cells overproducing 2HG. Zaprinast, a PDE5 inhibitor, was identified as a modulator of 2HG production. The ease of the fluorimetric assay in the high-throughput setting, coupled with its quantitative capacity and low cost, was encouraging from a drug discovery perspective. While a relatively modest library of 480 compounds was screened, interrogation of much larger compound libraries as well as siRNA libraries are also feasible by this method and provide important strategies to identify targets that reduce 2HG for therapeutic purposes and define mechanisms of regulation.

Among the other compounds identified were a calcium channel modulator and a PKC inhibitor. Further follow up was performed with Zaprinast because the drug showed the most dramatic reduction in 2HG levels and several other PDE5 inhibitors have undergone clinical approval. Also, several PKC inhibitors have undergone clinical trial development for use in cancer, heart failure, coronary artery disease, and diabetic retinopathy (Mochly-Rosen et al., 2012). Without being bound by theory, the effects of Zaprinast are likely not mediated by cGMP, as neither the cell-permeable cGMP analogue, 8-bromo-cGMP, nor expression of constitutively active sGC reduced 2HG levels. Instead, a previously undescribed off-target effect of Zaprinast was found against GLS1, the first enzyme of glutaminolysis, which ultimately supplies αKG for mutant IDH reactions.

Losman et al. recently showed that withdrawal of cell-permeable 2HG following transformation induced by treatment of cell-permeable 2HG restores dependence on growth factors and differentiation of leukemia cells (Losman et al., 2013). Similarly, treatment with small molecule inhibitors specifically targeting IDH1 R132H and IDH2 R140Q showed that many of the effects of mutant IDH, including histone hypermethylation, colony formation, and differentiation, were indeed reversible (Rohle et al., 2013; Wang et al., 2013). Indirectly blocking 2HG production by inhibiting GLS1 (and thus flux through mutant IDH1) was capable of reversing histone hypermethylation and soft agar growth in human astrocytes, indicating that indirectly blocking mutant IDH1 activity by inhibiting GLS1 may serve as an alternative therapeutic strategy in appropriately reprogrammed cells. This is of particular interest with regard to mutant IDH1-induced histone hypermethylation since inhibition of GLS1 simultaneously reduced both cellular 2HG and αKG. In the face of opposing metabolite actions (2HG inhibits αKG-dependent dioxygenases such as the JmjC histone demethylases, while αKG activates), histone hypermethylation states were nonetheless reversed overall by Zaprinast treatment, suggesting that the activity of histone demethylases could be more sensitive to reductions in 2HG than αKG. Zaprinast is the first small molecule inhibitor not directly targeting mutant IDH to show this effect and broadens the targets in the pathway amenable to modulation. Selective targeting of GLS1 may be important for subsets of patients with IDH1 or IDH2 mutations not affected by small molecule inhibitors developed against target-specific IDH mutations or patients displaying resistance to IDH1-targeted therapies.

Interestingly, Seltzer et al. (2010) showed that GLS1 inhibition by BPTES preferentially limits growth of D54 glioblastoma cells expressing IDH1 R132H when compared to wild type IDH1. However, 2HG levels were unaffected by BPTES. Without being bound by theory, this may be attributed to differences in metabolic wiring that allows some cells to overcome GLS1 inhibition. Indeed, Cheng et al. showed that some cells can utilize pyruvate carboxylase for anapleurosis under conditions of glutamine deprivation or silencing of GLS1 (Cheng et al., 2011). Thus, it is possible that differences in expression of pyruvate carboxylase may dictate responsiveness to pharmacological inhibition of GLS1 in IDH1 mutant tumors.

GLS1 expression has been shown to correlate with tumor growth and tumor grade independent of IDH mutations (Cassago et al., 2012). c-Myc-driven expression of GLS1 promotes glutaminolysis and glutamine dependence, and furthermore, inhibition of GLS1 was shown to cause a marked reduction in growth of several glutamine-dependent cell lines and xenograft models (Wise et al., 2008; Gao et al., 2009; Wang et al., 2010). Son et al. showed that PDAC cells rely on glutamine metabolism for maintenance of redox state (Son et al., 2013). Here, the effect of Zaprinast on Panc1 and MiaPaca2 cells was examined and it was shown that Zaprinast inhibits cell growth, caused an increase in ROS levels, and sensitized cells to oxidative damage by hydrogen peroxide, all in a manner that can be significantly rescued by extracellular glutamate. Thus, Zaprinast mimics the effects of glutamine deprivation in PDAC cells, and points to a targeted small molecule strategy amenable to translation.

Thus, this highlights the potential utility of pharmacologically targeting GLS1 in cancer and further stresses the need for potent and safe inhibitors (Wang et al., 2010; Shukla et al., 2012). A new chemical structure has been identified that is capable of directly inhibiting the activity of purified GLS1 noncompetitively, which typically implies allosteric binding of a compound to a target enzyme. Importantly, treatment of multiple independent cell lines with Zaprinast altered cellular metabolite levels in a manner most consistent with GLS1 inhibition as the mechanism of action, rather than another off-target activity. In addition, in Zaprinast-treated cells, addition of cell permeable glutamate rescued growth and abolished the heightened sensitivity to oxidative damage, providing further evidence for a GLS1-targeted mechanism in live cells.

### II. IDH MUTANT CANCER

IDH1 and IDH2 are the cytoplasmic and mitochondrial NADP⁺-dependent isocitrate dehydrogenases, respectively, and are homologs. IDH3, which is unrelated to IDH1 and IDH2, is the NAD⁺-dependent isocitrate dehydrogenase and has not been found to be mutated in cancer. These enzymes convert isocitrate to α-ketoglutarate. IDH1 catalyzes this reaction in the cytosol and peroxisome to mediate a variety of cellular housekeeping functions, while IDH2 and IDH3 catalyze a step in the TCA cycle. IDH1 R132 mutations occur frequently in astrocytomas and oligodendrogliomas, as well as in secondary glioblastomas and may be the initiating lesion in these glioma subtypes. Mutations in the analogous IDH2 R172 codon also occur, but at a lower rate, in these cancers. Mutations in IDH1 and IDH2 have also been observed in acute myelogenous leukemias. In gliomas, R132H is the most common IDH1 mutation, and R172K is the most common IDH2 mutation. IDH1 and IDH2 mutations are mutually exclusive and alter only one allele, apparently in a dominant fashion. Mutation of these codons abolishes the normal ability of IDH1 and IDH2 to convert isocitrate to α-ketoglutarate. IDH1 R132 and IDH2 R172 mutants gain the ability to convert α-ketoglutarate to 2HG, which is highly elevated in IDH-mutated cancer tissues. Increased levels of 2-HG are thought to promote carcinogenesis by competitively inhibiting enzymes that use α-ketoglutarate as a cofactor.

Mutations in the gene encoding cytosolic NADP+-dependent isocitrate dehydrogenase (IDH1) emerged as an unsuspected finding in sequence analysis of glioblastoma (GBM) (Parsons, 2008). Recent studies reported on mutations in the IDH1 gene resulting in an amino acid exchange in position 132 in about 70% of anaplastic gliomas and 12% of glioblastomas (Balls, 2008; Yan, 2009; Watanabe, 2009; De Carli, 2009; Hartmann, 2009; Ichimura, 2009; Sanson, 2009). IDH1 mutations also occur in a high frequency in WHO grade II and III diffuse gliomas, and 93% of all IDH1 mutations are characterized by an amino acid exchange R132H (Hartmann, 2009). In addition, R132 has been found to be mutated to Cys, Ser, Glu, and Lys. In addition to R132, amino acid positions G97, R100, H133, and Y139 are known to be mutated.

Provided herein are methods of treating a cancer characterized by the presence of a mutant allele of IDH1 or IDH2 comprising the step of administering to a subject in need thereof (a) a compound described in any one of the embodiments herein, or a pharmaceutically acceptable salt thereof, or (b) a pharmaceutical composition comprising (a) and a pharmaceutically acceptable carrier.

IDH mutations have been identified in glioblastoma, acute myelogenous leukemia, sarcoma, melanoma, non-small cell lung cancer, cholangiocarcinomas, chondrosarcoma, myelodyspiasiic syndromes (MDS), myeloproliferative neoplasm (MPN), colon cancer, and angio-immunoblastic non-Hodgkin's lymphoma (NHL). Accordingly, in one embodiment, the methods described herein are used to treat glioma (glioblastoma), acute myelogenous leukemia, sarcoma, melanoma, non-small cell lung cancer (NSCLC) or cholangiocarcinomas, chondrosarcoma, myelodyspiasiic syndromes (MDS), myeloproliferative neoplasm (MPN) or colon cancer in a patient. Accordingly, the cancer may be a cancer selected from any one of the cancer types listed.

In one embodiment, the cancer to be treated may be characterized by a mutant allele of IDH1 wherein the IDH1 mutation results in a new ability of the enzyme to catalyze the NAPH⁺-dependent reduction of α-ketoglutarate to 2-hydroxyglutarate in a subject. In one aspect, the mutant IDH1 may have an R132X mutation. In one aspect, the R132X mutation may be selected from R132H, R132C, R132L, R132V, R132S and R132G. In another aspect, the R132X mutation may be R132H or R132C. In yet another aspect, the R132X mutation may be R132H. In other aspects, the mutant IDH1 may have a mutation at G97, R100, H133, or Y139. A cancer can be analyzed by sequencing cell samples to determine the presence and specific nature of a mutation at (e.g., the changed amino acid present at) amino acid 97, 100, 132, 133, or 139 of IDH1.

In one embodiment, the cancer to be treated may be characterized by a mutant allele of IDH2 wherein the IDH2 mutation results in a new ability of the enzyme to catalyze the NAPH⁺-dependent reduction of α-ketoglutarate to 2-hydroxyglutarate in a subject. In one aspect, the mutant IDH2 may have an R172X mutation. In one aspect, the R172X mutation may be selected from R172M, R172K, R172G, and R172W. In another aspect, the R172X mutation may be R172M. In other aspects, the mutant IDH1 may have a mutation at R140, and the mutation may be a R140Q, R140W, or R140L substitution. A cancer can be analyzed by sequencing cell samples to determine the presence and specific nature of (e.g., the changed amino acid present at) a mutation at amino acid 140 or 172 of IDH2.

In one embodiment, prior to, during, and/or after treatment with a compound described in any one of the embodiments described herein, the method further comprises the step of evaluating the IDH1 and/or IDH2 genotype of the cancer. This may be achieved by ordinary methods in the art, such as DNA sequencing, immuno analysis, and/or evaluation of the presence, distribution, or level of 2HG.

The IDH1 or IDH2 gene, transcript, and protein may be detected in cultured cells or cells isolated from a mammal using any of the methods described in the instant application or those well known in the art. In some cases, the presence or absence of an IDH1 or IDH2 mutation can be detected by assessing the gene sequence or transcript of the gene. For example, an IDH1 and IDH2 gene may be detected by Southern blot, PCR, sequencing, a peptide nucleic acid-locked nucleic acid clamp method, Northern blot, RT-PCR, and the like. In some cases, the presence or absence of an IDH1 or IDH2 mutation can be detected by assessing the protein sequence, expression levels, and/or distribution. For example an IDH1 and IDH2 protein may be detected by immunohistochemistry, Western blot, mass spectrometry, and the like.

In one embodiment, prior to, during, and/or after treatment with a compound described in any one of the embodiments described herein, the method may further comprise the step of determining the 2HG level in the subject. This may be achieved by spectroscopic analysis, e.g., magnetic resonance-based analysis, e.g., MRI and/or MRS measurement, sample analysis of bodily fluid, such as serum or spinal cord fluid analysis, or by analysis of surgical material, e.g., by mass-spectroscopy.

In other words, the efficacy of cancer treatment may be monitored by measuring the levels of 2HG in the subject. Levels of 2HG may be measured prior to treatment, wherein an elevated level may be indicative of the use of a compound of described in any one of the embodiments described herein to treat the cancer. Once elevated levels are established, the level of 2HG may be determined during the course of and/or following termination of treatment to establish efficacy. In certain embodiments, the level of 2HG may only be determined during the course of and/or following termination of treatment. A reduction of 2HG levels during the course of treatment and following treatment may be indicative of efficacy. Similarly, a determination that 2HG levels are not elevated during the course of or following treatment may be also indicative of efficacy. 2HG measurements may be utilized together with other well-known determinations of efficacy of cancer treatment, such as reduction in number and size of tumors and/or other cancer-associated lesions, improvement in the general health of the subject, and alterations in other biomarkers that are associated with cancer treatment efficacy. As such, prior to, during, and/or after treatment with a compound described in any one of the embodiments described herein, the method may further comprise a step of evaluating the growth, size, weight, invasiveness, stage and/or other phenotype of the cancer.

2HG can be detected in a sample by LC/MS. For this, a sample is mixed 80:20 with methanol, and centrifuged at 3,000 rpm for 20 minutes at 4 degrees Celsius. The resulting supernatant can be collected and stored at −80° C. prior to LC-MS/MS to assess 2-hydroxy glutarate levels. A variety of different liquid chromatography (LC) separation methods can be used. Each method can be coupled by negative electrospray ionization (ESI, −3.0 kV) to triple-quadrupole mass spectrometers operating in multiple reaction monitoring (MRM) mode, with MS parameters optimized on infused metabolite standard solutions. Metabolites can be separated by reversed phase chromatography using 10 mM tributyl-amine as an ion pairing agent in the aqueous mobile phase, according to a variant of a previously reported method (Luo et al., 2007). One method allows resolution of TCA metabolites: t=0, 50% B; t=5, 95% B; t=7, 95% B; t=8, 0% B, where B refers to an organic mobile phase of 100% methanol. Another method is specific for 2-hydroxyglutarate, running a fast linear gradient from 50%-95% B over 5 minutes. A Synergi Hydro-RP, 100 mm×2 mm, 2.1 μm particle size (Phenomonex) can be used as the column. Metabolites can be quantified by comparison of peak areas with pure metabolite standards at known concentration. Metabolite flux studies from ¹³C-glutamine can be performed as described, e.g., in Munger et al. (2008).

### III. DIAGNOSTIC IMAGING

The compounds of the embodiments can be used in diagnostic compositions, such as probes, particularly when they are modified to include appropriate labels (e.g., heavy isotope labels). The administered compounds can be detected using known detection methods appropriate for the label used. Examples of detection methods include magnetic resonance imagining (MRI) of hyperpolarized compounds, position emission topography (PET) and single-photon emission computed tomography (SPECT). Specific imaging techniques that may be applied to the compositions and methods of the embodiments are detailed below.

Hyperpolarized MRI

The metabolic conversion of hyperpolarized heavy-isotope (e.g., ¹³C or ¹⁵N)-labeled glutamine and/or glutamate into α-ketoglutarate or 2HG can be used to study metabolic processes in the human body using magnetic resonance. The ratio of glutamine:glutamate and the amount of α-ketoglutarate and 2HG is dependent on the metabolic status of the tissue under investigation. In addition, hyperpolarized heavy-isotope labeled Zaprinast derivatives may be used to image target availability and engagement.

The terms “hyperpolarized” and “polarized” are used interchangeably hereinafter and denote a nuclear polarization level in excess of 0.1%, more preferred in excess of 1% and most preferred in excess of 10%.

The level of polarization may for instance be determined by solid state heavy isotope-NMR measurements with solid hyperpolarized compounds. The solid state heavy isotope-NMR measurement preferably consists of a simple pulse-acquire NMR sequence using a low flip angle. The signal intensity of the hyperpolarized heavy-isotope-labeled compound in the NMR spectrum is compared with signal intensity of heavy-isotope-labeled compound in a NMR spectrum acquired before the polarization process. The level of polarization is then calculated from the ratio of the signal intensities of before and after polarization.

In a similar way, the level of polarization for dissolved hyperpolarized heavy-isotope-labeled compounds may be determined by liquid state NMR measurements. Again the signal intensity of the dissolved hyperpolarized heavy-isotope-labeled compounds is compared with the signal intensity of the dissolved heavy-isotope-labeled compounds before polarization. The level of polarization is then calculated from the ratio of the signal intensities of heavy-isotope-labeled compounds before and after polarization.

The term “imaging medium” denotes a liquid composition comprising but not limited to a hyperpolarized heavy-isotope-labeled substance as the MR active agent. The imaging medium according to embodiments of the invention may be used as imaging medium in MR imaging or as MR spectroscopy agent in MR spectroscopy and MR spectroscopic imaging.

The imaging medium according to the method of embodiments of the invention may be used as imaging medium for in vivo MR imaging, spectroscopy and/or spectroscopic imaging, i.e. MR imaging, spectroscopy and/or spectroscopic imaging carried out on living human or non-human animal beings. Further, the imaging medium according to the method of embodiments of the invention may be used as imaging medium for in vitro MR imaging, spectroscopy and/or spectroscopic imaging, e.g., for detecting and monitoring the ratio of glutamine:glutamate and the flux through the glutamine-glutamate pathway. Cell cultures may be derived from cells obtained from samples derived from the human or non-human animal body like for instance blood, urine or saliva while ex vivo tissue may be obtained from biopsies or surgical procedures.

The isotopic enrichment of the hyperpolarized heavy-isotope labeled compounds used in the method of embodiments of the invention is preferably at least 75%, more preferably at least 80% and especially preferably at least 90%, an isotopic enrichment of over 90% being most preferred. Ideally, the enrichment is 100%.

Hyperpolarization of NMR active heavy-isotope-labeled-nuclei may be achieved by different methods which are for instance described in WO-A-98/30918, WO-A-99/24080 and WO-A-99/35508, which are incorporated herein by reference and hyperpolarization methods are polarization transfer from a noble gas, “brute force,” spin refrigeration, the parahydrogen method and dynamic nuclear polarization (DNP). Hyperpolarized glutamine can be made as described in WO2013/149935, which is incorporated herein by reference in its entirety.

As mentioned earlier, the imaging medium according to the method of embodiments of the invention may be used as imaging medium for in vivo MR imaging, spectroscopy, and/or spectroscopic imaging, i.e., MR imaging, spectroscopy, and/or spectroscopic imaging carried out on living human or non-human animal beings. Such an imaging medium preferably comprises, in addition to the MR active agent, an aqueous carrier, preferably a physiologically tolerable and pharmaceutically accepted aqueous carrier like water/saline, a buffer or a mixture of buffers. The imaging medium may further comprise conventional pharmaceutically acceptable carriers, excipients and formulation aids. Thus, the imaging medium may for example include stabilizers, osmolality adjusting agents, solubilizing agents and the like, e.g., formulation aids such as are conventional for diagnostic compositions in human or veterinary medicine.

Further, the imaging medium according to the method of embodiments of the invention may be used as imaging medium for in vitro MR imaging, spectroscopy, and/or spectroscopic imaging, e.g., for cell cultures or ex vivo tissues. Such an imaging medium preferably comprises, in addition to the MR active agent, a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

An MR imaging sequence is applied that encodes the volume of interest in a combined frequency and spatially selective way and the MR signal of heavy-isotope-labeled compound is followed by MR imaging or spectroscopic imaging over a time period from the addition of the imaging agent (t=0) to about 1 min or until the MR signal is undetectable due to the signal decay via T1 relaxation. In the same time period, the appearance, increase and/or decrease of the heavy-isotope-labeled metabolite signal is monitored. To get a quantitative assessment, MR imaging, spectroscopy, or spectroscopic imaging of healthy cells or tissue may carried out and the results—i.e. the amount or rate of metabolite formed over a given time period—may be compared.

If the hyperpolarized heavy-isotope-labeled compound is used as an imaging agent in an in vivo method of MR imaging, spectroscopy or spectroscopic imaging, e.g. in a living human or non-human animal body, the imaging medium containing the hyperpolarized heavy-isotope-labeled compound is preferably administered to said body parenterally, preferably intravenously. Generally, the body under examination is positioned in the MR magnet. Dedicated heavy isotope-MR RF-coils are positioned to cover the area of interest. Dosage and concentration of the imaging medium will depend upon a range of factors such as toxicity and the administration route. The administration rate is preferably less than 10 ml/s, more preferably less than 6 ml/s and most preferable of from 5 ml/s to 0.1 ml/s. At less than 400 s after the administration, preferably less than 120 s, more preferably less than 60 s after the administration, especially preferably 20 to 50 s an MR imaging sequence is applied that encodes the volume of interest in a combined frequency and spatial selective way. The exact time of applying an MR sequence is highly dependent on the volume of interest.

The encoding of the volume of interest can be achieved by using so-called spectroscopic imaging sequences, such as but not limited to those described in for instance Brown et al. (1982); Maudsley et al. (1983); Mayer et al. (2006); Kohler et al. (2007); Yen et al. (2009). Spectroscopic image data contain a number of volume elements in which each element contains a full heavy isotope-MR spectrum.

In another preferred embodiment, the imaging medium comprising hyperpolarized heavy-isotope-labeled compound is administered repeatedly, thus allowing longitudinal studies.

The results obtained in the method of embodiments of the invention for instance may allow the physician to choose the appropriate treatment for the patient under examination. In a further preferred embodiment, the method of embodiments of the invention may be used to determine whether treatment is successful.

### IV. METHODS OF TREATING

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a therapeutically effective amount of a compound described in any one of the embodiments herein to a cancer patient in need thereof.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

A. Compounds of the Present Invention

The compounds provided by the present disclosure are shown, for example, above in the summary of the invention section and in the claims below. The methods of making these compounds can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein. Additional descriptions of the methods to produce these compounds can be found in U.S. Pat. Nos. 5,521,191 and 6,156,753, which are both incorporated herein by reference.

Compounds of the invention may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the compounds provided herein are within the scope of the invention. It will also be appreciated by those skilled in organic chemistry that many organic compounds can exist in more than one crystalline form. For example, crystalline form may vary from solvate to solvate. Thus, all crystalline forms of the compounds provided herein or the pharmaceutically acceptable solvates thereof are within the scope of the present invention.

B. Pharmaceutical Formulations and Routes of Administration

Where clinical application of a therapeutic composition is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via intravenous, intramuscular, sub-cutaneous, or even intraperitoneal routes. Typically, such compositions can be prepared as either liquid solutions, suspensions, or dispersions; solid forms suitable for use to prepare solutions, suspensions, or dispersions upon the addition of a liquid prior to injection can also be prepared; and, the preparations can also be emulsified. Dispersions can be prepared in glycerol, liquid polyethylene glycols, and mixtures thereof and in oils. Under ordinary conditions of storage and use, these preparations may contain a preservative to prevent the growth of microorganisms. Compounds may also be administered by continuous perfusion/infusion of a disease or wound site. Compounds may also be administered orally, and the active compounds may be coated in a material to protect the compound from the action of acids and other natural conditions which may inactivate the compound.

Sterile injectable solutions can be prepared by incorporating the compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient (i.e., the therapeutic compound) plus any additional desired ingredient from a previously sterile-filtered solution thereof.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient (i.e., the therapeutic compound) plus any additional desired ingredient from a previously sterile-filtered solution thereof.

Compounds can be orally administered, for example, with an inert diluent or an assimilable edible carrier. The therapeutic compound and other ingredients may also be enclosed in a hard or soft shell gelatin capsule, compressed into tablets, or incorporated directly into the patient's diet. For oral therapeutic administration, the therapeutic compound may be incorporated, for example, with excipients and used in the form of ingestible tablets, buccal tablets, troches, capsules including hard or soft capsules, elixirs, emulsions, solid dispersions, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the patients to be treated, each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The actual dosage amount of a compound or composition comprising a compound administered to a patient may be determined by physical and physiological factors, such as age, sex, body weight, severity of condition, the type of disease being treated, previous or concurrent therapeutic interventions, idiopathy of the patient, and the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

An effective amount typically will vary from about 0.001 mg/kg to about 1000 mg/kg, from about 0.01 mg/kg to about 750 mg/kg, from about 100 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1000 mg per day. In some particular embodiments, the amount is less than 10,000 mg per day with a range of 750 mg to 9000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day, or less than 10 mg/kg/day. It may alternatively be in the range of 1 mg/kg/day to 200 mg/kg/day. In some embodiments, the amount could be 10, 30, 100, or 150 mg/kg formulated as a suspension in sesame oil. In some embodiments, the amount could be 3, 10, 30 or 100 mg/kg administered daily via oral. In some embodiments, the amount could be 10, 30, or 100 mg/kg administered orally. For example, regarding treatment of diabetic patients, the unit dosage may be an amount that reduces blood glucose by at least 40% as compared to an untreated patient. In another embodiment, the unit dosage is an amount that reduces blood glucose to a level that is ±10% of the blood glucose level of a non-diabetic patient.

In other non-limiting examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 μg/kg body weight to about 500 mg/kg body weight, etc., can be administered, based on the numbers described above.

Single or multiple doses of the agent comprising a compound are contemplated. Desired time intervals for delivery of multiple doses can be determined by one of ordinary skill in the art employing no more than routine experimentation. As an example, patients may be administered two doses daily at approximately 12 hour intervals. In some embodiments, the agent is administered once a day. The agent(s) may be administered on a routine schedule. As used herein a routine schedule refers to a predetermined designated period of time. The routine schedule may encompass periods of time that are identical or that differ in length, as long as the schedule is predetermined. For instance, the routine schedule may involve administration twice a day, every day, every two days, every three days, every four days, every five days, every six days, a weekly basis, a monthly basis, or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

C. Combination Therapy

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both a compound of the present embodiments and a second therapy. A tissue, tumor, or cell can be contacted with one or more compositions or pharmacological formulation(s) comprising one or more of the agents (i.e., a compound of the present embodiments or a second anti-cancer agent), or by contacting the tissue, tumor, and/or cell with two or more distinct compositions or formulations, wherein one composition provides 1) a compound of the present embodiments, 2) a second anti-cancer agent, or 3) both a compound of the present embodiments and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

A compound of the present embodiments may be administered before, during, after, or in various combinations relative to an anti-cancer treatment. The administrations may be in intervals ranging from concurrently to minutes to days to weeks. In embodiments where a compound of the present embodiments is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with a compound of the present embodiments and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a compound of the present embodiments therapy is “A” and a second anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

i. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

ii. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

iii. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

iv. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

v. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double or may represent a dative or coordination bond to a metal atom. The symbol “ [symbol] ” represents a single bond or a double bond. Thus, for example, the formula

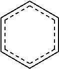

includes

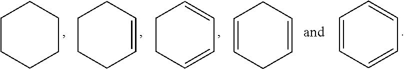

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

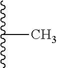

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. The bond orders described above are not limiting when one of the atoms connected by the bond is a metal atom (M). In such cases, it is understood that the actual bonding may comprise significant multiple bonding and/or ionic character. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

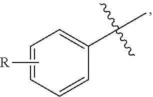

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

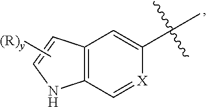

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms. (Cn-n′) defines both the minimum (n) and maximum number (n′) of carbon atoms in the group. Similarly, “alkyl_{(C2-10)}” designates those alkyl groups having from 2 to 10 carbon atoms.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl, with the carbon atom that forms the point of attachment also being a member of one or more non-aromatic ring structures wherein the cycloalkyl group consists of no atoms other than carbon and hydrogen. As used herein, the term does not preclude the presence of one or more alkyl groups (carbon number limitation permitting) attached to the ring or ring system. The groups —CH₃ (Me), —CH₂CH₃ (Et), —CH₂CH₂CH₃ (n-Pr or propyl), —CH(CH₃)₂ (i-Pr, ⁱPr or isopropyl), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

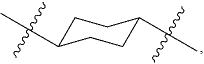 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

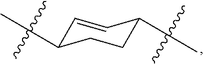 [SMILES: *C1C=CC(*)CC1]

are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —CH═CHF, —CH═CHCl and —CH═CHBr, are non-limiting examples of substituted alkenyl groups.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. An “alkyne” refers to the compound H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

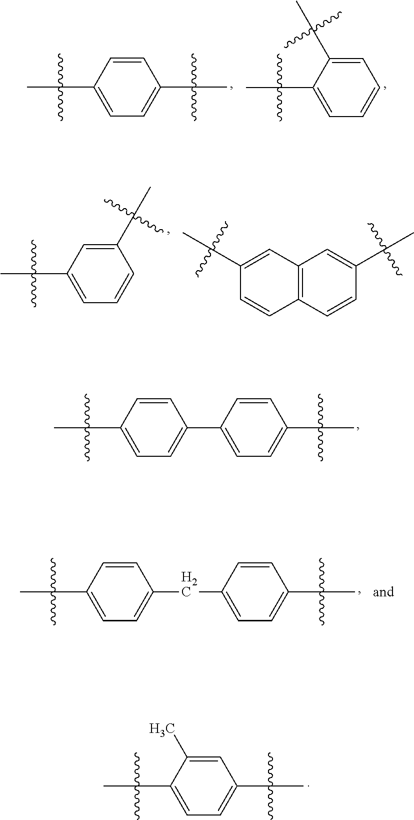

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

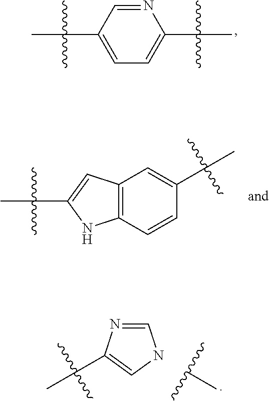

A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of heteroaralkyls are: pyridinylmethyl and 2-furanyl-propyl. When the term heteroaralkyl is used with the “substituted” modifier one or more hydrogen atom from the alkanediyl and/or the heteroaryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. Non-limiting examples of substituted heteroaralkyls are: (3-chloropyridinyl)-methyl, and 2-chloro-2-imidazolyl-but-1-yl.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “alkenyloxy”, “alkynyloxy”, “aryloxy”, “aralkoxy”, “heteroaryloxy”, “heteroaralkyloxy”, and “acyloxy”, when used without the “substituted” modifier, refers to groups, defined as —OR, in which R is alkenyl, alkynyl, aryl, aralkyl, heteroaryl, heteroaralkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

As used herein, average molecular weight refers to the weight average molecular weight (Mw) as determined by static light scattering.

As used herein, a “chiral auxiliary” refers to a removable chiral group that is capable of influencing the stereoselectivity of a reaction. Persons of skill in the art are familiar with such compounds, and many are commercially available.

The terms “comprise” and “include” are open-ended linking verbs. Any forms or tenses of one or more of these verbs, such as “comprises,” “comprising,” “includes,” and “including,” are also open-ended. For example, any method that “comprises” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating a disease, is sufficient to effect such treatment for the disease. The time between baseline study prior to initiation of therapy and time of evaluation of efficacy can vary but would typically fall in the range of 4-12 weeks, 12-24 weeks, or 24-52 weeks.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Plasmid construction. IDH1 cDNA clone (BC012846.1) was purchased from ATCC in the pCMV-Sport6 backbone. Site-directed mutagenesis was carried out to introduce a g395a mutation (R132H) and sequence verified. The ORF of both wild type and R132H IDH1 were then subcloned into pcDNA 3.1 and pLVX-IRES-Hyg vectors using standard molecular biology techniques. Histidine-6× tagged E. coli 3-phosphoglycerate dehydrogenase (PHGDH) plasmid was a kind gift from Dr. Gregory Grant (Washington University School of Medicine). sGCα and sGCβ_{cys}105 plasmids were a kind gift from Dr. Emil Martin (University of Texas Health Sciences Center, Houston). Human GLS1 cDNA (BC126537) was purchased from Thermo-Scientific and the ORF was subcloned into pSV281 containing an N-terminal 6× His tag using forward and reverse restriction sites, BamH1 and HindIII, respectively. pLKO.1-puro shRNA constructs were provided by the Washington University Genome Institute and used for RNA interference against IDH1. Sequences for the shRNAs were as follows: 1) 5′-CCTTTGTATCTGAGCACCAAA-3′ (SEQ ID NO: 1) and 2) 5′-GCTGCTTGCATTAAAGGTTTA-3′ (SEQ ID NO: 2).

Cell culture, transfection, and generation of stable cell lines. HT1080, HEK293T, Panc1, and MIA PaCa-2 cells were obtained from the American Type Culture Collection (ATCC). Normal human astrocytes (NHA) immortalized with E6/E7/hTERT were a kind gift from Dr. Russell O. Pieper (University of California, San Francisco). All cells tested negative for mycoplasma infection. HT1080 cells were authenticated by genotyping for IDH1 and were confirmed to harbor a heterozygous IDH1 R132C mutation by Sanger sequencing of genomic DNA products. The remaining cell lines were not further authenticated. HEK293T, NHA, PANC-1, and MIA Paca-2 cells were maintained with complete DMEM containing 10% fetal bovine serum (FBS) and 1% penicillin/streptomycin (P/S). HT1080 cells were maintained in αMEM containing 10% FBS and 1% P/S. Glutamine-free DMEM (Life Technologies) was supplemented with 10% FBS and 1% P/S and spiked with varying amounts of L-glutamine (Cellgro) for glutamine growth curves. Zaprinast (Sigma-Aldrich) and H₂O₂ were added to the appropriate fresh media as indicated.

For production of lentivirus, 1×10⁶ HEK293T cells were co-transfected with pCMV-VSVS-G, pCMVAR8.2, and either pLKO.1-puro for RNA interference experiments or pLVX-IRES-Hyg constructs for stable over-expression using Fugene® 6 (Promega) for 48 hours, after which viral supernatants were collected and transferred to cells of interest. Cells transduced with pLKO.1-euro were then selected in batch with 1 μg/mL puromycin for 72 hours, while cells transduced with pLVX-IRES-Hyg were selected in batch with 100 μg/mL hygromycin for 1 week.

2HG fluorimetric assay. N-terminal 6×-His tagged PHGDH was expressed and purified from chemically competent BL-21 E. coli using Ni-NTA agarose (Qiagen) (Grant et al., 1999). Purified protein was then dialyzed in 40 mM KPO₄ buffer at 4° C. overnight. Conditioned culture media (DMEM or aMEM) containing 2HG was neutralized with 60 mM HCL for 10 minutes followed by 60 mM Tris base. A 20 μL aliquot of neutralized conditioned media was then mixed with 90 μL of optimized assay reaction mix consisting of 5 μg purified PHGDH enzyme, 12.5 μM resazurin (Sigma-Aldrich), 0.125 U/ml diaphorase (Sigma-Aldrich), 1 mM NAD (Sigma-Aldrich), 40 mM Tris-HCl pH 8.8, and incubated for 90 minutes in a 96-well plate. Resorufin fluorescence was then quantified using a FLUOstar™ OPTIMA fluorescence plate reader (BMG Labtech) with excitation and emission peaks of 544 nm and 590 nm, respectively. Measurements were background subtracted (neutralized fresh media) and normalized either to total protein or viability as measured by Alamar Blue (Naik et al., 2009).

High-throughput screening. Screening was carried out using the Beckman Coulter (Fullerton, Calif.) Core Robotics system, including an FX liquid handler, controlled by the Sagian graphical method development tool (SAMI scheduling software). The Institute of Chemistry and Cell Biology (ICCB) Known Bioactives Library (Enzo Lifescience, BML-2840-0100) was diluted 1:200 in αMEM and 50 μL were added to pre-plated HT1080 cells. After 48 hours of incubation, media was removed and 2HG in the media was measured using the fluorimetric assay. Fresh 2HG fluorimetric assay reaction mix was prepared immediately before assaying each screen plate to limit background modulation. Cell viability was determined using Alamar Blue, as described previously (Naik et al., 2009).

To identify screen hits and account for differences in compound toxicity, 2HG fluorescence was first median-centered around DMSO treatment controls within each screen plate. 2HG fluorescence was then plotted against viability such that a correlation between the two measurements could be visualized. A linear fit and 95% predictive interval (P.I.) were then determined and plotted using GraphPad Prism (GraphPad Software).

Animal studies. All animal experiments were approved by the Institutional Animal Care and Use Committee at Washington University in St. Louis School of Medicine. To generate tumor xenografts, 8-week-old nu/nu mice (Taconic) were injected subcutaneously in the flank with 3×10⁶ HT1080 cells in 100 μL αMEM. Tumors were allowed to grow for 11 days before treatments were started. Intratumoral injection was performed on days 11, 13, and 15 with 20 μL of solution containing vehicle (DMSO) or Zaprinast (600 μM) (Sigma-Aldrich). Tumor volume was calculated as l×w×h of tumor dimensions obtained by caliper measurements. On day 15, tumors were extracted and snap frozen for follow up GC-MS analysis.

Histone extraction and Western blot analysis. For extraction of total cellular protein, cells were lysed in RIPA buffer (150 mM NaCl, 50 mM Tris, 5 mM EDTA, 1% NP-40, 0.1% SDS, 0.5% sodium deoxycholate, pH 7.4), supplemented with protease inhibitor cocktail (Roche), sodium orthovanadate (1 mM), and PMSF (1 mM). Whole-cell lysates were normalized for protein content by BCA assay (Pierce). Histone extraction was performed using an EpiQuik™ Total Histone Extraction Kit (Epigentek) and extracted protein was quantified using a Bradford assay.

Proteins were resolved by SDS-PAGE, transferred to a PVDF membrane and probed with the following antibodies: IDH1 (Origene, TA500610), 6× His (Abcam, ab1187), H3K9Me2 (Cell Signaling, 4658p), H3K9Me3 (Abcam, ab8898), H3K27Me3 (Millipore, 07-449), H3K79Me2 (Cell Signaling, 9757p), total H3 (Cell Signaling, 4499p), and Actin. Secondary anti-mouse and anti-rabbit horseradish peroxidase-conjugated IgG antibodies were used for detection.

GC-MS analysis. Metabolite extraction from cultured cells was performed as described previously by Figueroa et al. (2010). Briefly, cells were rinsed in ice cold PBS and rapidly quenched with 80% methanol spiked with 3-hydroxy-1,5-pentanedioic-2,2,3,4,4-d5 acid (CDN Isotopes, Quebec Canada) as an internal standard. Extracts were then incubated at −80° C. for 20 minutes, sonicated on ice, centrifuged at 14,000×g for 20 minutes at 4° C. to clear precipitate proteins, and supernatants transferred to vials for drying under N₂. Derivatizing reagent (MSTFA (N-methyl-N-(trimethylsilyl)trifluoroacetamide):pyridine:acetonitrile 1:1:2) was added to the vials, which were then heated at 70° C. for 15 minutes.

Derivatized samples were analyzed on an Agilent 7890A gas chromatograph interfaced to an Agilent 5975C mass spectrometer. The GC column used for the study was a HP-5MS (30 m, 0.25 mm i.d., 0.25 μm film coating). A linear temperature gradient was used. The initial temperature of 80° C. was held for 2 minute and increased to 300° C. at 10° C./minute. The temperature was held at 300° C. for 2 minutes. The samples were run by electron ionization (EI) and the source temperature, electron energy, and emission current were 200° C., 70 eV, and 300 μA, respectively. The injector and transfer line temperatures were 250° C. 3-Hydroxy-1,5-pentanedioic-2,2,3,4,4-d5 acid was used as the internal standard in SIM mode for quantitation of 2-hydroxyglutaric acid. Quantitation was carried out by monitoring the ions at m/z 347 (glutamine and αKG), 348 (glutamate), 349 (2-hydroxyglutaric acid), and 354 (3-hydroxy-1,5-pentanedioic-2,2,3,4,4-d5).

Glutaminase assay. N-terminal 6×-His tagged GLS1 was expressed and purified from chemically competent BL-21 E. coli using Ni-NTA agarose beads (Qiagen). Purified protein was then dialyzed in a 50 mM Tris-phosphate buffer containing 1 mM dithiothreitol at 4° C. overnight. A previously described two-step assay was used to measure glutaminase activity (Kenny et al., 2003). Briefly, 30 μL of purified glutaminase (660 μg/ml) were incubated with various concentrations of Zaprinast at 37° C. for 15 minutes. Zaprinast was dissolved in DMSO and 3 μL was added to each glutaminase aliquot to maintain a constant concentration of DMSO across samples. Initial reaction mix (30 μl) containing 20 mM glutamine, 0.15 M KPO₄, 0.2 mM EDTA, and 50 mM Tris-acetate (pH 8.6) was then added and samples were incubated at 37° C. for 10 minutes after which the reaction was rapidly quenched with 6 μL of 3 N HCl. To generate blank samples, glutaminase and Zaprinast solutions were inactivated with 3 N HCl before the addition of initial reaction mix. For the second step of the assay, 20 μL of completed and quenched initial reaction were transferred to a new plate and 200 μL of secondary reaction mix containing 0.4 mg bovine liver glutamate dehydrogenase (Sigma-Aldrich), 0.09 M Tris-acetate (pH 9.4), 0.2 M hydrazine, 0.25 mM ADP, and 2 mM NAD was added. Samples were incubated for 40 minutes at room temperature and absorbance of NADH (340 nm) was measured using a microplate spectrophotometer.

Quantification of intracellular cGMP. HT1080 cells were rinsed with ice cold PBS, lysed with 0.1 mM HCl, and centrifuged at 1,000×g for 10 minutes. Supernatant was then assayed for cGMP using a cGMP Enzyme Immunoassay kit (Cayman Chemical, 581021) according to the manufacturer's protocol.

ROS quantification. Reactive oxygen species levels in PDAC cells were quantified using 2,7-dichlorofluorescein diacetate (DCFDA). Following 48 hours of drug treatment in a 96-well plate, cells were washed with PBS, stained for 30 minutes at 37° C. with 20 μM DCFDA, and imaged using an InCell Analyzer 1000. A 10× objective was used to collect 16 fluorescent and bright field images per well with a 10% overlap to allow image stitching. Stained cells were then quantified using the GE InCell Investigator software package with Developer Toolbox.

Soft agar assay. A bottom layer of 0.6% soft agar with complete DMEM and 10% FBS was set in 6-well plates and allowed to solidify. Approximately 8,000 cells were then suspended in 0.4% soft agar and plated on top. Colonies were allowed to develop over 3-4 weeks and stained with crystal violet.

Statistical analysis. Statistical significance was evaluated using Student's t-test or two-way ANOVA as indicated. Error bars represent S.D. unless otherwise indicated.

### Example 1

### Novel Coupled Fluorescence Assay for Quantitation of 2HG

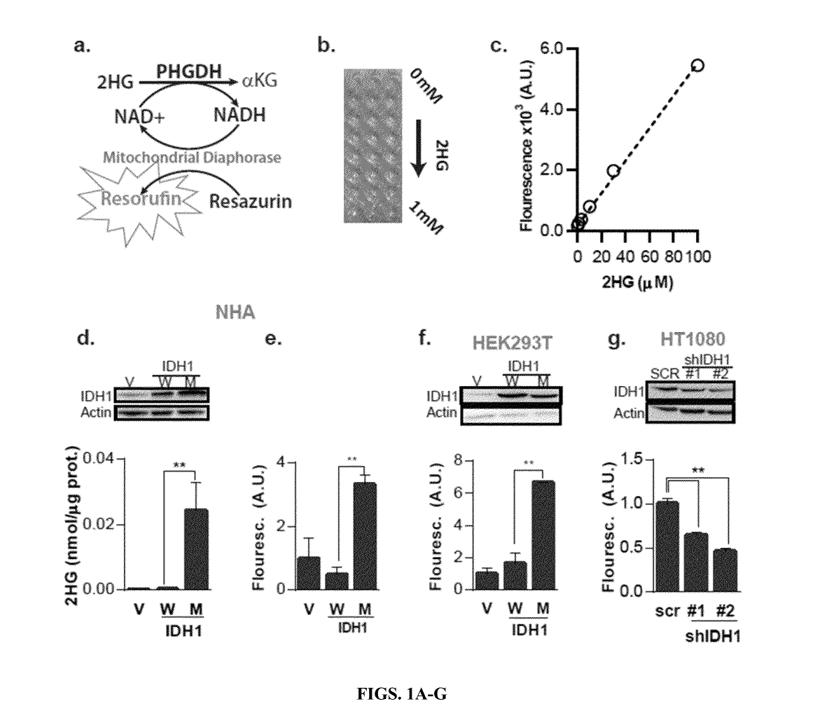

Conventional methods to measure 2HG in cell culture rely on time consuming and costly chromatography platforms that are not amenable to high throughput screening experiments. Instead, an enzymatically-coupled fluorescence assay was developed to directly quantify the total levels of 2HG in cultured cells. The strategy involved a primary enzymatic reaction that is specific to 2HG and produces NADH, which is coupled to a second reaction that consumes NADH with mitochondrial diaphorase. The E. coli K-12 enzyme 3-phosphoglycerate dehydrogenase (PHGDH) is known to have dehydrogenase activity against human R-2HG and uses NAD+ as a co-factor (Zhao and Winkler, 1996). Published reaction conditions for the 2HG dehydrogenase activity of E. coli PHGDH provided a basis for its use in the fluorescence assay. The NADH produced by E. coli PHGDH is subsequently oxidized by the diaphorase enzyme to reduce resazurin to resorufin, a highly fluorescent red-shifted molecule with an emission peak at 587 nm, thereby minimizing auto-fluorescence (FIG. 1A). In the process, NADH is recycled back to NAD⁺ and resorufin is produced in stoichiometric proportion to the amount of 2HG present in the sample (Zhu et al., 2009). The assay is rapid, inexpensive, quantitative and non-destructive if measuring secreted metabolites, making it ideal for large scale screening applications.

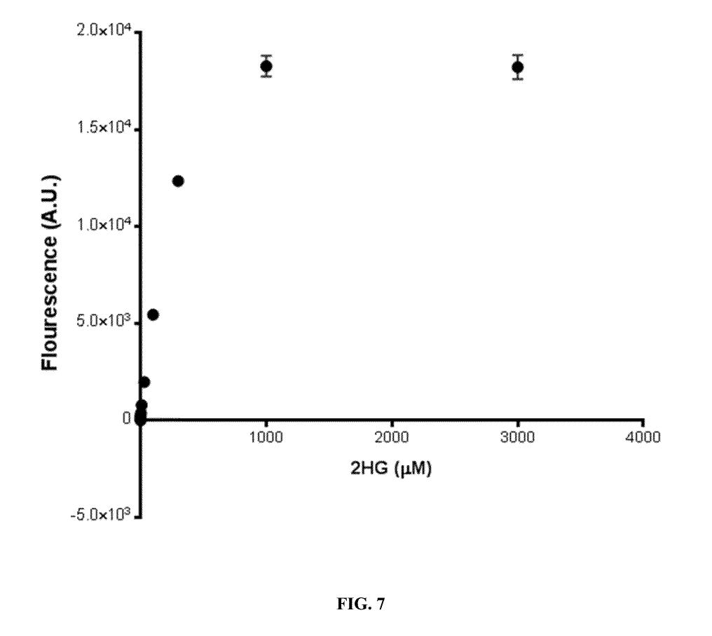

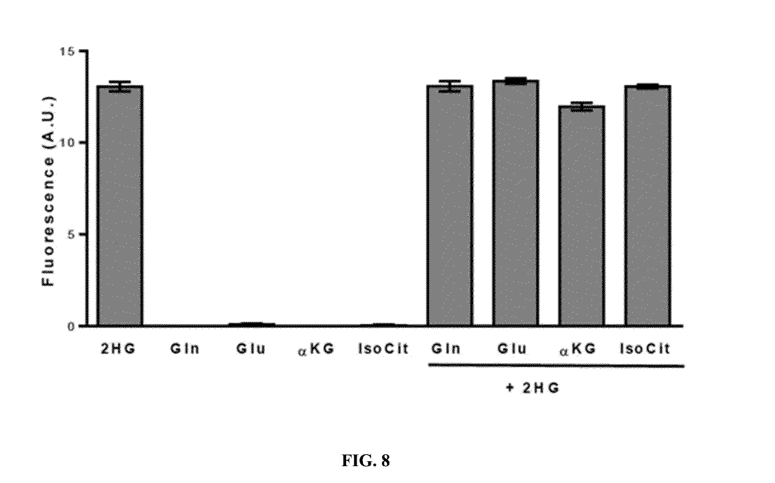

After optimization of assay conditions, a change was detected in fluorescent signal in direct proportion to added 2HG concentrations (FIGS. 1B and C). Exogenous 2HG was detected when dissolved in culture media in a concentration-dependent manner, indicating that secreted 2HG could be both detected and quantified (FIG. 1C). However, signal background was measurably higher in the fluorescent assay when compared to mass spectrometry. By assaying a 20 μL aliquot of culture media, assay activity was determined to be linear to 100 μM and saturated at 2HG concentrations of 1 mM or higher; the lower limit of detection was approximately 4 μM (FIG. 7). The specificity of the assay for 2HG was determined by testing the effect of metabolites within the IDH metabolic pathway, including glutamine, glutamate, αKG, and isocitrate and it was found that they do not interfere with assay function (FIG. 8). Next, to determine if the assay could detect physiological changes in 2HG production by cultured cells, immortalized human astrocytes were stably transduced with vector, wild-type IDH1, or R132H IDH1. Accumulation of 2HG in astrocytes expressing R132H IDH1 was first confirmed by conventional GC-MS (FIG. 1D). Then, by the fluorimetric assay, media from astrocytes expressing mutant IDH1 showed significantly higher signal when compared to cells expressing either vector or wild type IDH1 (FIG. 1E). This was also verified in HEK293T cells (FIG. 1F). In addition, to determine if the fluorimetric assay could detect a reduction in 2HG levels, shRNA knockdown of IDH1 was performed in the human fibrosarcoma cell line HT-1080, which harbors an endogenous heterozygous gain-of-function R132C IDH1 mutation. Using two different hairpin sequences, 2HG levels were assayed in the culture media and showed a significant reduction in signal in proportion to IDH1 protein knockdown (FIG. 1G).

### Example 2

### High-Throughput Screen for Modulators of 2HG Metabolism

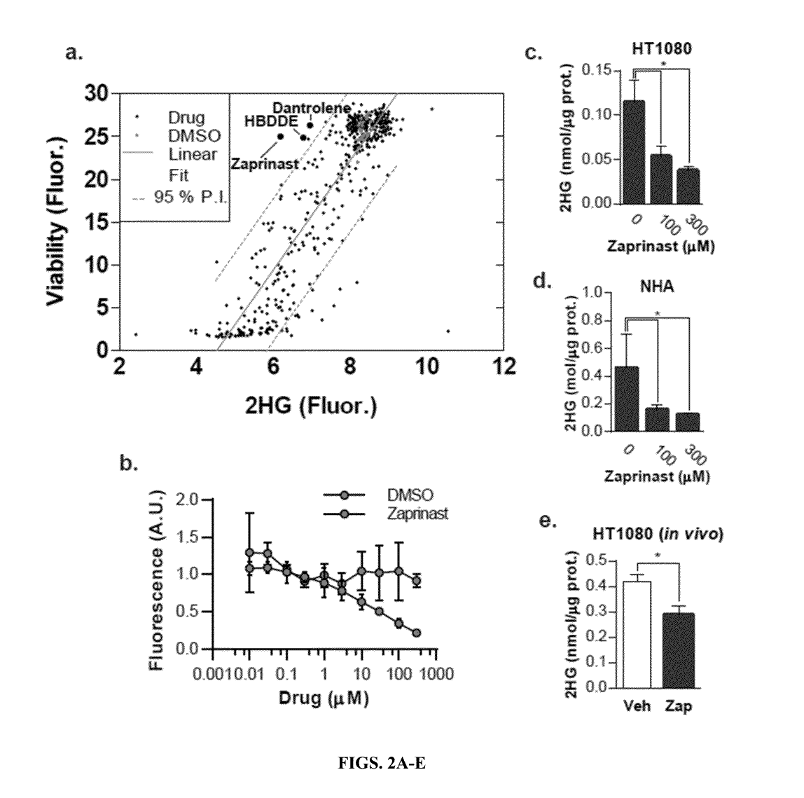

Using the above assay to quantify 2HG in media, a library of 480 bioactive compounds targeting a wide variety of cellular processes were screened in IDH1 mutant HT-1080 cells to identify targets capable of affecting the 2HG metabolic pathway. Screen results are plotted in FIG. 2A as 2HG fluorescence versus viability. A global linear trend was observed between 2HG and viability indicating that most 2HG reduction was driven by compound toxicity. However, screen hits in the top left quadrant reduced 2HG but had little effect on viability over the time course of the assay (FIG. 2A). Among the top candidate compounds were Zaprinast, a phosphodiesterase type 5 (PDE5) inhibitor, HBDDE, a PKC inhibitor, and Dantrolene, a calcium release inhibitor (FIG. 2A). Zaprinast (5-(2-propoxyphenyl)-1H-[1,2,3]triazolo[4,5-d]pyrimidin-7(4H)-one) was the most potent compound identified in the screen and showed a concentration-dependent reduction in extracellular 2HG (FIG. 2B). To confirm that the effects of Zaprinast were not an artifact caused by an inhibition of the assay itself (i.e., E. coli PHGDH or diaphorase activity), secondary validation using mass spectrometry in extracts of HT1080 and NHA cells ectopically expressing R132H IDH1 and treated with Zaprinast was performed. As anticipated, production of 2HG was modulated in a concentration-dependent manner (FIGS. 2C,D).

To determine if Zaprinast could lower 2HG concentrations in vivo, flank tumor xenografts of HT1080 cells were generated in nu/nu mice. Because the pharmacokinetics of Zaprinast for this application have not been characterized, direct intratumoral injection was performed to assure delivery of the compound. Tumors treated with Zaprinast showed a modest but significant reduction of 2HG levels relative to vehicle as quantified by GC-MS (FIG. 2E).

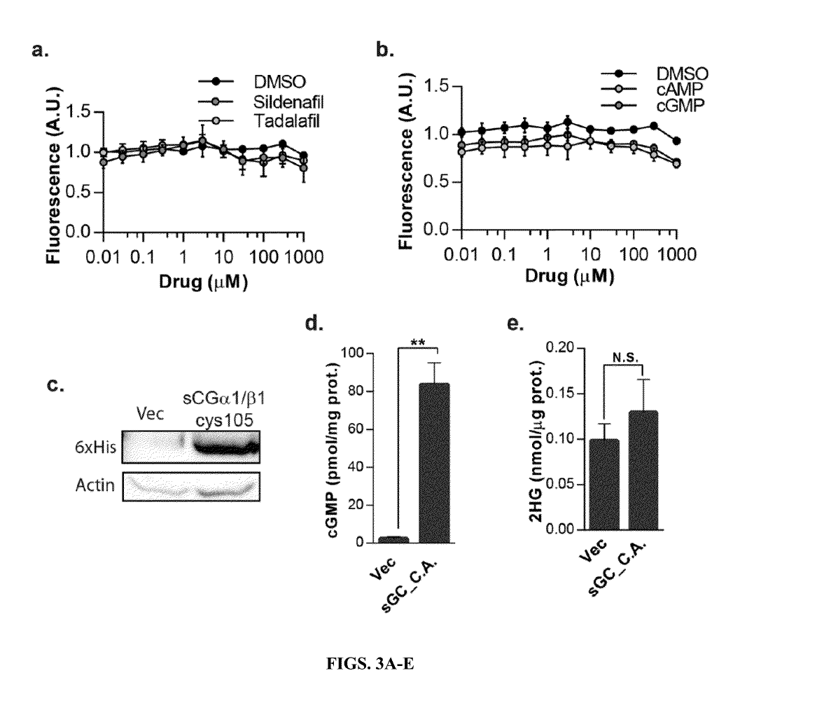

PDE5 hydrolyzes cGMP and is the target of several clinically-approved inhibitors that function by elevating intracellular cGMP (Sandner et al., 2007). However, no change in levels of 2HG were observed when treating HT1080 cells with Sildenafil and Tadalafil, two clinically-approved PDE5 inhibitors (FIG. 3A). Thus, it was determined if either cAMP or cGMP was sufficient to reduce 2HG levels in HT1080 cells. Treatment with the cell-permeable analogues 8-bromo-cAMP and 8-bromo-cGMP did not cause a reduction in 2HG (FIG. 3B). To confirm that the lack of 2HG reduction upon treatment with cell-permeable cGMP was not due to structural differences between the analogue and its endogenous counterpart, endogenous cGMP was elevated by expressing constitutively active soluble guanylyl-cyclase (sGC) (Martin et al., 2003). Co-expression of the wild type al subunit and the constitutively active β1 C105H mutant that comprise the sGC heterodimer resulted in a significant increase in basal cGMP levels as measured by ELISA (FIGS. 3C,D). 2HG levels, however, were unaffected by elevation of intracellular cGMP (FIG. 3E), indicating that the effects of Zaprinast on 2HG production were likely not a consequence of altered cGMP levels.

### Example 3

### Mechanism of Action of Zaprinast

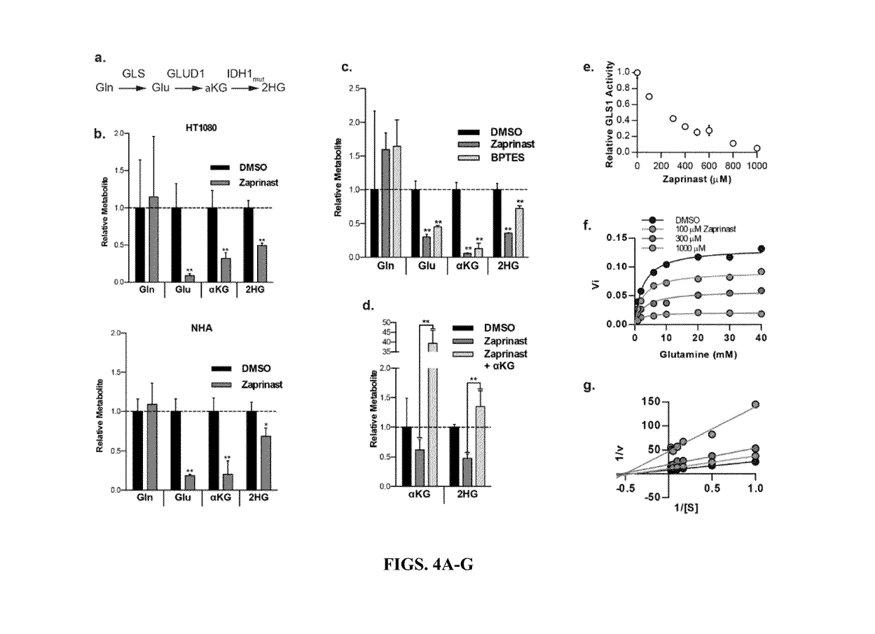

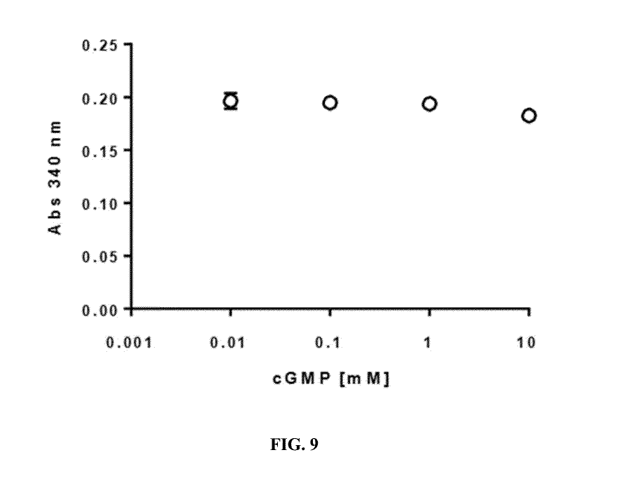

The reported IC₅₀ for Zaprinast against PDE5 is approximately 0.15 μM. However, approximately 100-300 μM were required to observe a significant reduction in 2HG. This suggested, along with the finding that the Zaprinast-mediated effects appeared to be cGMP-independent and not mimicked by other PDE5 inhibitors, that Zaprinast blocks 2HG production through an off-target effect. To define the mechanism of action of Zaprinast, changes in metabolites upstream of 2HG were examined. The predominant source of 2HG is cellular glutamine. Glutaminase (GLS1) metabolizes glutamine to glutamate, which is then converted to alpha-ketoglutarate by glutamate dehydrogenase, followed by mutant IDH1 metabolism of alpha-ketoglutarate to 2HG (FIG. 4A) (Dang et al., 2009). It was reasoned that by measuring upstream metabolites, one could identify a candidate target enzyme whose activity was either directly or indirectly inhibited by Zaprinast. Using mass spectrometry, cellular levels of glutamine, glutamate, alpha-ketoglutarate, and 2-hydroxyglutarate were measured in vehicle and Zaprinast-treated HT1080 cells. Unexpectedly, in addition to 2HG, levels of alpha-ketoglutarate and glutamate were also found to be reduced in HT1080 cells treated with Zaprinast, but intracellular levels of glutamine were unaffected. This was repeated in NHA immortalized astrocytes expressing R132H IDH1 and the same pattern of metabolite level changes were detected (FIG. 4B). These data suggested that Zaprinast directly or indirectly inhibited the activity of GLS1, causing a reduction in the level of downstream metabolites. Treatment of HT1080 cells with BPTES, a known GLS1 inhibitor, produced a similar pattern of metabolite modulation (FIG. 4C). Furthermore, in cells treated with Zaprinast, adding back a cell permeable form of alpha-ketoglutarate restored 2HG back to baseline levels (FIG. 4D), consistent with traversing a block at the level of GLS1. To determine if Zaprinast modulates 2HG by directly inhibiting GLS1, human GLS1 was purified and utilized in an in vitro assay to measure its enzymatic activity. The enzymatic activity of GLS1 was directly inhibited by Zaprinast with an IC₅₀ of ˜200 μM, which corresponded to the concentrations at which a significant reduction in 2HG was observed in cells (FIG. 4E). GLS1 kinetic analysis determined that Zaprinast acted as a noncompetitive inhibitor (Kᵢ=220 μM) (FIGS. 4F,G). In a control experiment, cGMP had no direct effect on GLS1 enzymatic activity (FIG. 9).

### Example 4

### Zaprinast-Mediated Modulation of the 2HG Phenotype

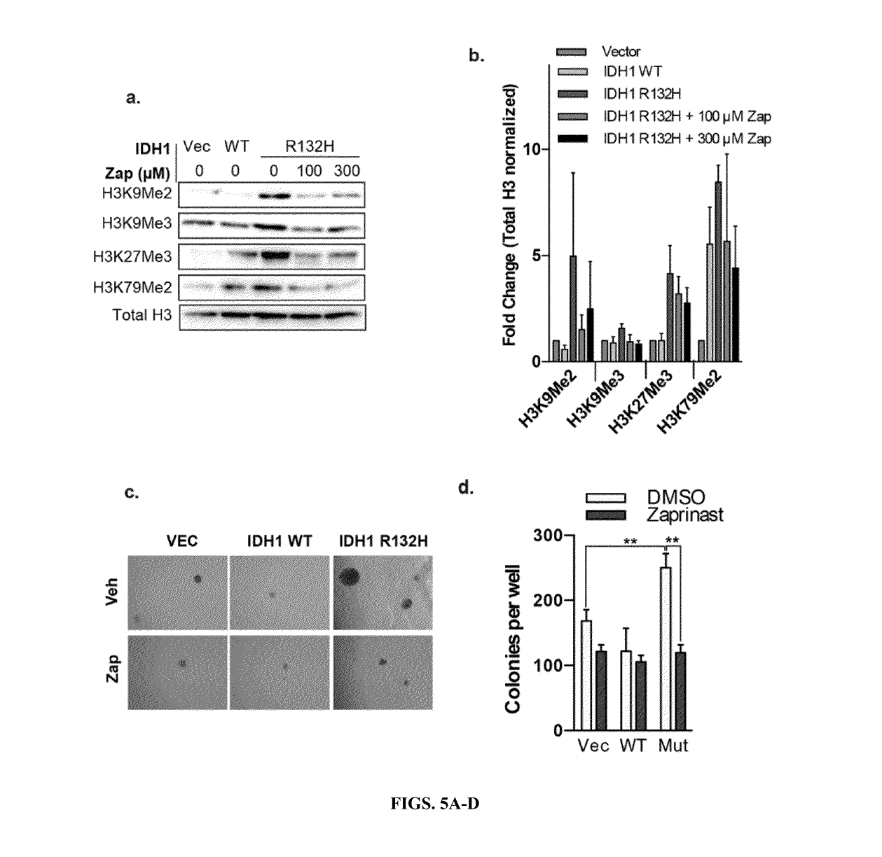

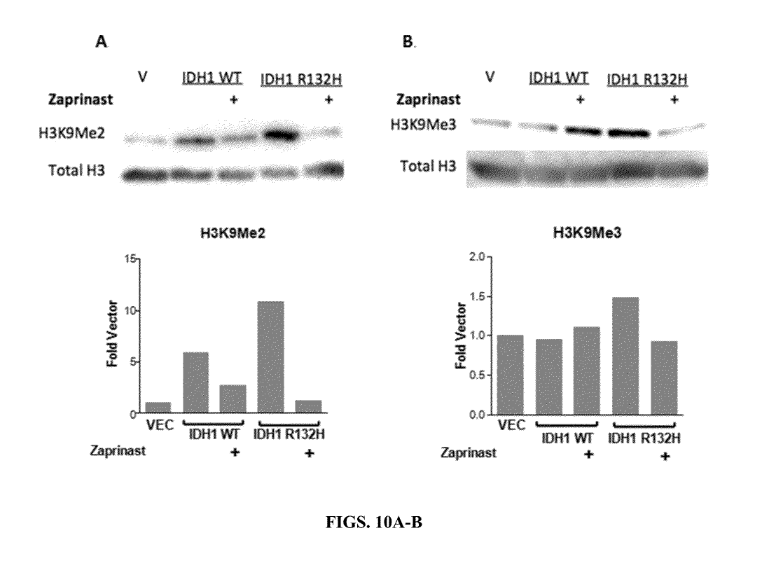

Since Zaprinast reduces 2HG levels by blocking flux through the pathway at the level of GLS1, it was next explored if Zaprinast treatment could reverse the effects of 2HG on cells. 2HG is structurally similar to αKG, differing only in the oxidation state of the C2 carbon (Losman and Kaelin, 2013). Because of this similarity, 2HG has been shown to act as either an inhibitor or activator of various αKG-dependent enzymes. The JmjC family of histone demethylases have been shown to be inhibited by 2HG and expression of mutant IDH1 was shown to cause an elevation in methylated histone lysine residues and lead to a block in cellular differentiation (Xu et al., 2011; Lu et al., 2012). As expected, expression of R132H IDH1 in human astrocytes caused an increase in histone methylation marks (H3K9Me2, H3K9Me3, H3K27Me3, and H3K79Me2), and treatment with Zaprinast caused a marked reduction toward baseline methylation levels (FIGS. 5A,B). In contrast, Zaprinast treatment of IDH1 WT expressing astrocytes did not produced such a marked reduction in methylation levels of either H3K9Me2 or H3K9Me3 (FIG. 10). In addition, mutant IDH1 was recently shown to promote soft agar colony formation of immortalized human astrocytes (Koivunen et al., 2012). As expected, IDH1 R132H expression increased colony formation of human astrocytes when compared to vector or IDH1 wild type. Importantly, Zaprinast treatment reduced colony formation in IDH1 R132H astrocytes down to the level of vector and wild-type cells (FIGS. 5C,D).

### Example 5

### Zaprinast Inhibits Glutamine-Dependent Tumor Cell Proliferation

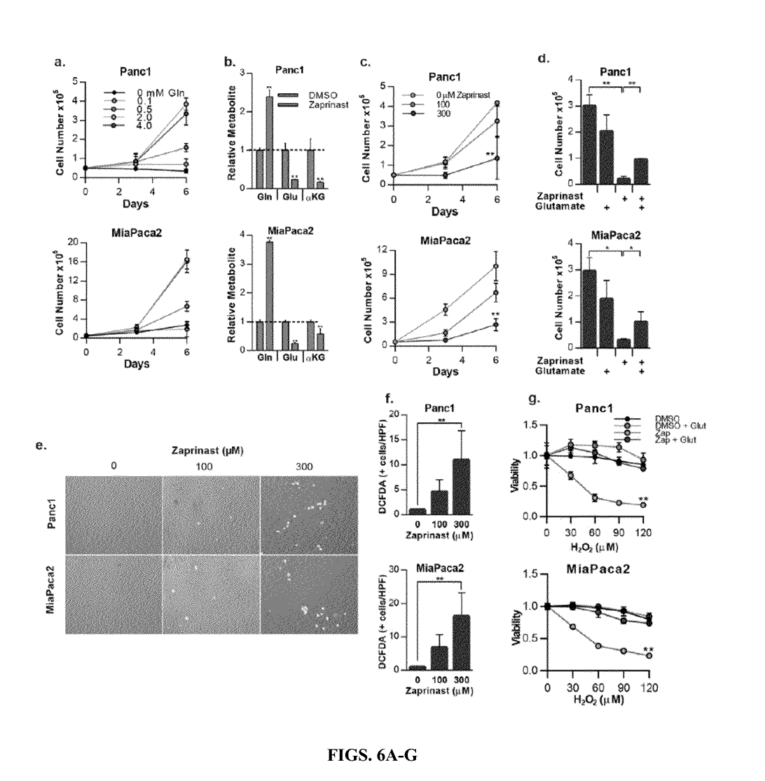

Because of the requirement for glutamine in 2HG production, it was hypothesized that disruption of glutamine metabolism with Zaprinast could abrogate the growth of glutamine-addicted cells with normal IDH activity. In cells undergoing the Warburg effect, whereby glucose is shunted toward lactate production in the presence of oxygen, glutamine often functions in anaplerotic reactions to replenish citric acid cycle intermediates (DeBerardinis et al., 2007). A recent report by Son et al. (2013) showed that HRAS-driven pancreatic ductal adenocarcinoma (PDAC) cells use glutamine in a different manner. PDAC cells are dependent on glutamine metabolism to maintain redox balance by shuttling glutamine carbon through the transaminase GOT1 and ultimately malic enzyme (ME) (Son et al., 2013). Using two PDAC cell lines, Panc1 and MiaPaca2, which are glutamine-addicted, but show minimally-detectable levels of 2HG, it was sought to determine if Zaprinast could perturb redox balance in a manner similar to glutamine deprivation. First, Panc1 and MiaPaca2 cells were confirmed to be dependent on extracellular glutamine for their growth, saturating at glutamine concentrations of 2 mM (FIG. 6A). Furthermore, treatment with Zaprinast led to significant reductions in cellular pools of glutamate and αKG and an increase in glutamine as measured by mass spectrometry, indicating that Zaprinast blocks GLS1 activity in live PDAC cells (FIG. 6B). Interestingly, glutamine levels were increased more dramatically in PDAC cells treated with Zaprinast when compared to NHA or HT1080 cells treated with Zaprinast (FIG. 4B). This is likely a consequence of RAS-driven metabolic reprogramming in PDAC cells wherein glutamine metabolic fluxes are high to maintain oxidative balance and promote transformation. Growth in the presence of 100 μM and 300 μM Zaprinast was significantly reduced (FIG. 6C). To confirm that growth inhibition in the presence of Zaprinast was due to GLS1 inhibition, glutamate supplementation of the media was performed, producing a restoration of growth (FIG. 6D). Consistent with previous findings that glutamine metabolism maintains oxidative homeostasis in PDAC, Zaprinast treatment of PDAC cells caused an increase in reactive oxygen species (ROS) in a concentration-dependent manner (FIGS. 6E,F). This resulted in increased susceptibility to oxidative damage as cells pretreated with Zaprinast were significantly more susceptible to hydrogen peroxide stress than cells treated with vehicle (FIG. 6G). Moreover, adding back glutamate to Zaprinast-treated cells nearly completely abolished the increased sensitivity to oxidative damage (FIG. 6G), again consistent with blockade at the level of GLS1. These findings suggest that Zaprinast could increase the sensitivity of PDAC to oxidative stress, as might be therapeutically induced by radiation or chemotherapy.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,156,753
U.S. Pat. No. 5,521,191
PCT Publn. No. WO98/30918
PCT Publn. No. WO99/24080
PCT Publn. No. WO99/35508
PCT Publn. No. WO2013/149935
Amary et al., IDH1 and IDH2 mutations are frequent events in central chondrosarcoma and central and periosteal chondromas but not in other mesenchymal tumours. J Pathol, 224:334-343, 2011.
Balls, Acta Neuropathol, 116:597-602, 2008.
Balss et al., Enzymatic assay for quantitative analysis of (D)-2-hydroxyglutarate. Acta Neuropathol, 124:883-891, 2012.
Borger et al., Frequent mutation of isocitrate dehydrogenase (IDH)1 and IDH2 in cholangiocarcinoma identified through broad-based tumor genotyping. Oncologist, 17:72-79, 2012.
Brown et al., Proc Natl Acad Sci USA, 79:3523-3526, 1982.
Cassago et al., Mitochondrial localization and structure-based phosphate activation mechanism of Glutaminase C with implications for cancer metabolism. Proc Natl Acad Sci USA, 109:1092-1097, 2012.
Cheng et al., Pyruvate carboxylase is required for glutamine-independent growth of tumor cells. Proc Natl Acad Sci USA, 108:8674-8679, 2011.
Dang et al., Cancer-associated IDH1 mutations produce 2-hydroxyglutarate. Nature, 462:739-744, 2009.
DeBerardinis and Cheng, Q's next: the diverse functions of glutamine in metabolism, cell biology and cancer. Oncogene, 29:313-324, 2010.
DeBerardinis et al., Beyond aerobic glycolysis: transformed cells can engage in glutamine metabolism that exceeds the requirement for protein and nucleotide synthesis. Proc Natl Acad Sci USA, 104:19345-19350, 2007.
De Carli, N Engl J Med, 360:2248, 2009.
Figueroa et al., Leukemic IDH1 and IDH2 mutations result in a hypermethylation phenotype, disrupt TET2 function, and impair hematopoietic differentiation. Cancer Cell, 18:553-567, 2010.
Gao et al., c-Myc suppression of miR-23a/b enhances mitochondrial glutaminase expression and glutamine metabolism. Nature, 458:762-765, 2009.
Grant et al., The relationship between effector binding and inhibition of activity in D-3-phosphoglycerate dehydrogenase. Protein Sci, 8:2501-2505, 1999.
Hartmann, Acta Neuropathol, 118:469-474, 2009.
Ichimura, Neuro Oncol, 11:341-347, 2009.
Kenny et al., Bacterial expression, purification, and characterization of rat kidney-type mitochondrial glutaminase. Protein Expression and Purification, 31:140-148, 2003.
Kohler et al., Magn Reson Med, 58:65-69, 2007.
Koivunen et al., Transformation by the (R)-enantiomer of 2-hydroxyglutarate linked to EGLN activation. Nature, 483:484-488, 2012.
Losman and Kaelin, What a difference a hydroxyl makes: mutant IDH, (R)-2-hydroxyglutarate, and cancer. Genes Dev, 27:836-852, 2013.
Losman et al., (R)-2-hydroxyglutarate is sufficient to promote leukemogenesis and its effects are reversible. Science, 339:1621-1625, 2013.
Lu et al., IDH mutation impairs histone demethylation and results in a block to cell differentiation. Nature, 483:474-478, 2012.
Luo et al., J Chromatogr A, 1147:153-64, 2007.
Mardis et al., Recurring mutations found by sequencing an acute myeloid leukemia genome. N Engl J Med, 361:1058-1066, 2009.
Martin et al., A constitutively activated mutant of human soluble guanylyl cyclase (sGC): implication for the mechanism of sGC activation. Proc Natl Acad Sci USA, 100:9208-9213, 2003.
Maudsley et al., J Magn Res, 51:147-152, 1983.
Mayer et al., Magn Reson Med, 56:932-937, 2006.
Mochly-Rosen et al., Protein kinase C, an elusive therapeutic target? Nat Rev Drug Discov, 11:937-957, 2012.
Munger et al., Nat Biotechnol, 26:1179-86, 2008.
Naik et al., Vascular Endothelial Growth Factor Receptor-1 Is Synthetic Lethal to Aberrant {beta}-Catenin Activation in Colon Cancer. Clin Cancer Res, 15:7529-7537, 2009.
Ongur et al., Relationship between genetic variation in the glutaminase gene GLS1 and brain glutamine/glutamate ratio measured in vivo. Biol. Psychiatry, 70:169-174, 2011.
Parsons et al., An integrated genomic analysis of human glioblastoma multiforme. Science, 321:1807-1812, 2008.
Rohle et al., An inhibitor of mutant IDH1 delays growth and promotes differentiation of glioma cells. Science, 340:626-630, 2013.
Sandner et al., PDE5 inhibitors beyond erectile dysfunction. Int J of Impotence Research, 19:533-543, 2007.
Sanson, J Clin Oncol, 27:4150-4154, 2009.
Sasaki et al., IDH1(R132H) mutation increases murine haematopoietic progenitors and alters epigenetics. Nature, 488:656-659, 2012.
Seltzer et al., Inhibition of glutaminase preferentially slows growth of glioma cells with mutant IDH1. Cancer Res, 70:8981-8987, 2010.
Shukla et al., Design, synthesis, and pharmacological evaluation of bis-2-(5-phenylacetamido-1,2,4-thiadiazol-2-yl)ethyl sulfide 3 (BPTES) analogs as glutaminase inhibitors. J Med Chem, 55:10551-10563, 2012.
Son et al., Glutamine supports pancreatic cancer growth through a KRAS-regulated metabolic pathway. Nature, 496:101-105, 2013.
Terunuma et al., MYC-driven accumulation of 2-hydroxyglutarate is associated with breast cancer prognosis. J. Clin. Invest., 124:398-412, 2014.
Wang et al., Targeted inhibition of mutant IDH2 in leukemia cells induces cellular differentiation. Science, 340:622-626, 2013.
Wang et al., Targeting mitochondrial glutaminase activity inhibits oncogenic transformation. Cancer Cell, 18:207-219, 2010.
Watanabe, Am J Pathol, 174:1149-1153, 2009.
Wise et al., Myc regulates a transcriptional program that stimulates mitochondrial glutaminolysis and leads to glutamine addiction. Proc Natl Acad Sci USA, 105:18782-18787, 2008.
Xu et al., Oncometabolite 2-hydroxyglutarate is a competitive inhibitor of alpha-ketoglutarate-dependent dioxygenases. Cancer Cell, 19:17-30, 2011.
Yan et al., IDH1 and IDH2 mutations in gliomas. N Engl J Med, 360:765-773, 2009.
Yen et al., Magn Reson Med, 62:1-10, 2009.
Zhao and Winkler, A novel alpha-ketoglutarate reductase activity of the serA-encoded 3-phosphoglycerate dehydrogenase of Escherichia coli K-12 and its possible implications for human 2-hydroxyglutaric aciduria. J Bacteriol, 178:232-239, 1996.
Zhu et al., An enzymatic fluorimetric assay for glucose-6-phosphate: application in an in vitro Warburg-like effect. Anal Biochem, 388:97-101, 2009.

1. A compound of the formula: 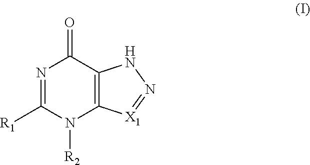
wherein:
R₁ and R₂ are each independently selected from hydrogen, alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, haloalkoxy_{(C≦12)}, alkenyloxy_{(C≦12)}, alkynyloxy_{(C≦12)}, aryloxy_{(C≦12)}, aralkyloxy_{(C≦12)}, heteroaryloxy_{(C≦12)}, heteroaralkyloxy_{(C≦12)}, acyloxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or 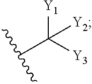
wherein:
Y₁ and Y₂ are independently selected from alkoxy_{(C≦8)} or substituted alkoxy_{(C≦8)} or Y₁ and Y₂ are taken together and are alkoxydiyl_{(C≦8 )} or substituted alkoxydiyl_{(C≦8)}; and
Y₃ is hydrogen, alkyl_{(C≦8)}, or substituted alkyl_{(C≦8)}; and
X₁ is N or CR₃;
R₃ is selected from hydrogen, alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, acyl_{(C≦12)}, alkoxy_{(C≦12)}, haloalkoxy_{(C≦12)}, alkenyloxy_{(C≦12)}, alkynyloxy_{(C≦12)}, aryloxy_{(C≦12)}, aralkyloxy_{(C≦12)}, heteroaryloxy_{(C≦12)}, heteroaralkyloxy_{(C≦12)}, acyloxy_{(C≦12)}, -alkanediyl_{(C≦8)}-alkoxy_{(C≦8)}, -alkanediyl_{(C≦6)}-arenediyl_{(C≦8)}-alkoxy_{(C≦8)}, -arenediyl_{(C≦12)}-alkoxy_{(C≦8)}, or a substituted version of any of these groups; or 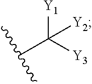
wherein:
Y₁ and Y₂ are independently selected from alkoxy_{(C≦8)} or substituted alkoxy_{(C≦8)}, or Y₁ and Y₂ are taken together and are alkoxydiyl_{(C≦8)} or substituted alkoxydiyl_{(C≦8)}; and
Y₃ is hydrogen, alkyl_{(C≦8)}, or substituted alkyl_{(C≦8)};
or a pharmaceutically acceptable salt, tautomer, acetal, or ketal thereof,
wherein the compound is not

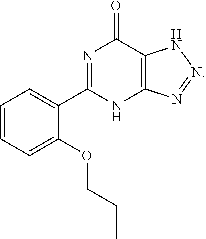 [SMILES: CCCOc1ccccc1-c1nc(=O)c2[nH]nnc2[nH]1]

2-14. (canceled)

15. A compound of claim 1 or having the formula: 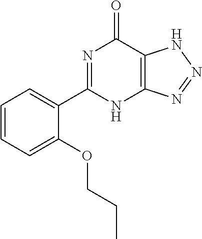 [SMILES: CCCOc1ccccc1-c1nc(=O)c2[nH]nnc2[nH]1]
wherein the compound comprises a heavy-isotope-label.

16. The compound of claim 15, wherein one or more positions of the compound are substituted with ¹³C or ¹⁵N.

17. An imaging composition comprising a compound of claim 15, in a pharmaceutically acceptable carrier.

18. A method of imaging a patient comprising:
(i) administering a composition comprising a labeled compound according to claim 17 to the patient; and
(ii) detecting the compound in the patient to produce an image.

19. (canceled)

20. A method of treating cancer in a patient in need thereof comprising administering to the patient a therapeutically effective amount of a compound of claim 1 or Zaprinast.

21. The method of claim 20, wherein the patient has been identified as having a cancer that comprises an IDH1 or IDH2 mutation.

22. The method of claim 20, further comprising selecting a patient determined to comprise a cancer comprising an IDH1 or IDH2 mutation prior to the administering step.

23. (canceled)

24. The method of claim 21, wherein the IDH1 or IDH2 mutation is a gain-of-function mutation in the IDH1 or IDH2 protein.

25. The method of claim 21, wherein the IDH1 mutation is a mutation at amino acid 100 or 132 of the IDH1 protein.

26. The method of claim 25, wherein the mutation at amino acid 132 of the IDH1 protein is selected from the group consisting of R132H, R132C, R132S, R132G, and R132L.

27. The method of claim 21, wherein the IDH2 mutation is a mutation at amino acid 140 or 172 of the IDH2 protein.

28. The method of claim 27, wherein the mutation at amino acid 140 or 172 of the IDH2 protein is selected from the group consisting of R140Q, R172M, R172K, and R172G.

29. (canceled)

30. The method of claim 20, wherein the patient has been identified as having a cancer with elevated levels of 2-hydroxyglutarate (2HG), a cancer that overexpresses glutaminase, or a cancer that comprises hyperactivated glutaminse.

31-36. (canceled)

37. The method of claim 20, wherein the cancer is a glioma, glioblastoma, acute myeloid leukemia, cholangiocarcinoma, chondrosarcoma, breast cancer, lung cancer, colorectal cancer, or pancreatic cancer.

38-48. (canceled)

49. A method of inhibiting glutaminase in a cell comprising treating the cell with a compound of claim 1 or Zaprinast.

50. A method of selecting a drug therapy for a cancer patient comprising:
(a) obtaining a sample of the cancer;
(b) determining the presence of a mutation in the IDH1 or IDH2 protein expressed in the cancer; and if a mutation is determined to be present in the IDH1 or IDH2 protein expressed in the cancer, then
(c) selecting a compound of claim 1 or Zaprinast.

51-53. (canceled)

54. A method of selecting a drug therapy for a cancer patient comprising:
(a) determining the flux through the glutamine:glutamate pathway; and
(b) selecting a compound of claim 1 or Zaprinast if the flux is determined to be higher than a reference level.

55-56. (canceled)

57. A method of determining the flux within the glutamine:glutamate pathway comprising performing hyperpolarized MR imaging, wherein the imaging agent is a heavy-isotope-labeled glutamine or glutamate.

58. A method of treating a patient with a psychiatric disorder comprising administering to the patient a therapeutically effective amount of a compound of claim 1 or Zaprinast.

59-61. (canceled)


SEQUENCE LISTING (2 sequences)

SEQ ID NO: 1 - DNA, 21 bp, Artificial Sequence
  Synthetic polynucleotide
cctttgtatctgagcaccaaa

SEQ ID NO: 2 - DNA, 21 bp, Artificial Sequence
  Synthetic polynucleotide
gctgcttgcattaaaggttta